SEQUENCE LISTING (18 sequences)

SEQ ID NO: 1 - DNA, 19 bp, Artificial sequence
  Synthetic Oligonucleotide
ggagaagtgcttgaagatt

SEQ ID NO: 2 - DNA, 19 bp, Artificial sequence
  Synthetic Oligonucleotide
cgtatcagtctggactcca

SEQ ID NO: 3 - DNA, 19 bp, Artificial sequence
  Synthetic Oligonucleotide
cgtatcagtctggactccg

SEQ ID NO: 4 - DNA, 23 bp, Artificial sequence
  Synthetic Oligonucleotide
gactacagcaaagatgcccagtg

SEQ ID NO: 5 - DNA, 20 bp, Artificial sequence
  Synthetic Oligonucleotide
gggatacttgggtggaagta

SEQ ID NO: 6 - DNA, 24 bp, Artificial sequence
  Synthetic Oligonucleotide
catatttattttgtccttctccca

SEQ ID NO: 7 - DNA, 18 bp, Artificial sequence
  Synthetic Oligonucleotide
gaaagtttccaccaacgg

SEQ ID NO: 8 - DNA, 18 bp, Artificial sequence
  Synthetic Oligonucleotide
gaaagtttccacgaacgc

SEQ ID NO: 9 - DNA, 19 bp, Artificial sequence
  Synthetic Oligonucleotide
gtgcagaaatgcaggaaat

SEQ ID NO: 10 - DNA, 21 bp, Artificial sequence
  Synthetic Oligonucleotide
gtgccagatattatttgtagg

SEQ ID NO: 11 - DNA, 27 bp, Artificial sequence
  Synthetic Oligonucleotide
cgaagcttgctgtttcttagggacacg

SEQ ID NO: 12 - DNA, 27 bp, Artificial sequence
  Synthetic Oligonucleotide
gtgaattccagaagggctcagagtggt

SEQ ID NO: 13 - DNA, 12 bp, Homo sapiens
gcgaacacctca

SEQ ID NO: 14 - DNA, 12 bp, Homo sapiens
  misc_feature (7)..(7): n = a or g
gcgaacncctca

SEQ ID NO: 15 - DNA, 12 bp, Homo sapiens
gcgaacgcctca

SEQ ID NO: 16 - DNA, 22 bp, Homo sapiens
acagcgaacacctcaggtctga

SEQ ID NO: 17 - DNA, 22 bp, Homo sapiens
acagcgaacgcctcaggtctga

SEQ ID NO: 18 - protein, 5 aa, Homo sapiens
  MISC_FEATURE (4)..(4): Xaa is Thr or Ala
TANXS

(19) United States
(12) Patent Application Publication
(10) Pub. No.: US 2004/0191764 A1
(43) Pub. Date: Sep. 30, 2004

(54) Screens for altered immune response capability

(76) Inventors: Peter Beverley, Berkshire (GB); Elma Tchilian, Berkshire (GB); Tara Stanton, Oxford (GB); Walter Bodmer, Oxford (GB)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/753,754

(22) Filed: Jan. 8, 2004

Related U.S. Application Data
(63) Continuation-in-part of application No. 10/020,758, filed on Oct. 30, 2001, now abandoned.

(30) Foreign Application Priority Data
Jun. 14, 2001 (GB) GB 0114512.7

(51) Int. Cl. C12Q 1/70; C12Q 1/68

(52) U.S. Cl. 435/5; 435/6

(57) ABSTRACT

The invention relates to associations between genetic variation in the gene encoding CD45 and human disease and human immune responses. In particular the invention provides methods of screening human subjects for the presence of an “altered immune response capability”, which may in turn affect susceptibility to viral disease and/or autoimmune disease.

### RELATED APPLICATIONS

This application is a continuation-in-part of U.S. application Ser. No. 10/020,758 filed Oct. 30, 2001, now pending, the entire contents of which is hereby incorporated by reference.

### FIELD OF THE INVENTION

The invention relates to associations between genetic variation in the gene encoding CD45 and human disease and human immune responses. In particular the invention provides methods of screening human subjects for the presence of an “altered immune response capability”, which may in turn affect susceptibility to viral disease and/or autoimmune disease.

### BACKGROUND

The leucocyte common antigen CD45 is an abundant tyrosine phosphatase, expressed on all leucocytes (Trowbridge, I. S., and M. L. Thomas. 1994. Ann. Rev. Immunol. 12:85). The phosphatase activity of CD45 is essential for lymphocyte antigen receptor signal transduction. Both CD45 deficient mice (Kishihara, K. et al., 1993. Cell 74:143; Byth, K. et al. 1996. J. Exp. Med. 183:170) and humans (Kung, C. et al., 2000, Nature Medicine, 6: 343; Tchilian, E. Z. et al., 2001, J. Immunol., 166: 1308) are severely immunodeficient, with very few peripheral T lymphocytes and impaired T and B cell responses.

Multiple CD45 isoforms can be generated by alternative splicing of exons A, B, and C of the extracellular domain (Saga, Y. et al., 1986. Proc Natl Acad Sci USA, 83: 6940; Streuli, M. et al., 1987, J. Exp. Med., 166: 1548). In humans, naive T cells express high molecular weight CD45 isoforms, recognised by CD45RA monoclonal antibodies (mAbs), but activation of the cells results in a change to expression of low molecular weight isoforms, detected by a CD45RO mAb (Akbar, A. N., et al., 1988, J. Immunol., 140: 2171). These two major subsets of T lymphocytes, expressing CD45RA and CD45RO have been termed naive and memory cells, respectively.

Genetically determined abnormal CD45 splicing has been described in humans (Schwinzer, R., and K. Wonigeit, 1990, J. Exp. Med. 171:1803.). Activated or memory lymphocytes in these individuals continue to express both high and low molecular weight CD45 isoforms in contrast to the normal pattern of low molecular weight isoform expression. A C to G transversion at position 77 (C77G) in the fourth or A exon of the gene encoding CD45, has been shown to prevent the normal splicing of this exon in the affected individuals (Thude, H. et al., 1995, Eur. J. Immunol., 25: 2101; Zilch, C. F. et al., 1998, Eur. J. Immunol., 28: 22) by disrupting a strong exonic splicing silencer (Lynch, K. W. a. W., 2001, J. Biol. Chem).

The C77G polymorphism has been shown to correlate with development of multiple sclerosis in some families (Jacobsen, M. et al., 2000, Nat Genet, 26: 495), although other studies do not support such an association (Vorechovsky, I. et al., 2001, Nat Genet, 29, 22-23; Barcellos, L. F. et al., 2001, Nat Genet, 29, 23-24.

A further point mutation in exon A of CD45 (C59A) causing aberrant splicing has been identified, but appears to be relatively rare (Jacobsen, M. et al., 2002, Immunogenetics, 54, 158-163).

The present inventors have investigated the pattern of CD45 expression in HIV infection and have demonstrated a statistically significant association between the C77G mutation and HIV-1 infection.

Further observations made by the present inventors provide evidence that the C77G mutation may be a marker for general susceptibility to viral infection and/or a marker for disease severity following viral infection. Accordingly, the inventors have developed screens for determining susceptibility of human subjects to viral infection and/or identifying individuals pre-disposed to developing more severe disease following viral infection based on screening for the presence or absence of the C77G mutation at the protein, mRNA or genomic DNA level.

The present inventors have further identified a novel polymorphism A138G in exon 6 in the gene encoding CD45 with a very high prevalence in Japanese and Korean populations. The expression of various CD45 isoforms in PBMC of individuals homo- and heterozygous for the 138G variant allele was analysed and the results show that T cells in individuals carrying the 138G allele display altered cell surface CD45 isoform expression due to changes in alternative splicing. The results suggest that individuals with the 138G variant allele have an increased proportion of T cells with an activated, memory or effector phenotype, as determined by the increased proportion of CD45RO+ cells and reduced number of cells expressing the CD45 A, B and C isoforms. Analysis of exon 6 A138G and exon 4 C77G variants in different populations showed striking differences in the frequency of these mutations, suggesting effects of natural selection and drift.

The inventors have still further identified a novel CD45 mutation, denoted A54G, in exon 4 in the gene encoding CD45. This A to G transversion results in a Thr to Ala semiconservative amino acid substitution at position 19 of the CD45RA exon 4 isoform. The A54G mutation was identified in African Ugandan populations and was found with an increased frequency amongst HIV-seropositive individuals.

The inventors findings relating to different CD45 mutations indicate that CD45 mutations can be used as genetic markers of immune function or immune capability. Furthermore, the inventors conclude that mutations in CD45 can be classified in two groups.

The first group of CD45 mutations (Group I) are associated with altered splicing of the CD45 mRNA and an altered pattern of CD45 isoform expression on the T cell population, characterized as a reduction in the proportion of T cell population carrying only the CD45RO splice variant (i.e. a reduction in CD45RO+ T cells) in individuals carrying at least one mutant allele, as compared to individuals not carrying a mutant allele. Examples of such Group I CD45 mutations are C77G, C59A and A54G. Carriage of a mutant allele of a Group I mutation is generally observed to be associated with increased susceptibility to viral infection and/or a pre-disposition to developing severe disease following viral infection. Accordingly, the inventors have developed genetic screens for evaluating susceptibility to viral infection based on genotyping of Group I CD45 mutations, or on analysis of altered patterns of CD45 mRNA or protein expression associated with carriage of a Group I mutation.

The second group of CD45 mutations (Group II) are associated with altered splicing of the CD45 mRNA and an altered pattern of CD45 protein isoform expression on the T cell population, characterized as an increase in the proportion of the T cell population carrying only the CD45RO splice variant (i.e. an increase in CD45RO+ T cells) in individuals carrying at least one mutant allele, as compared to individuals not carrying a mutant allele. An example of a Group II mutation is A138G. Carriage of a mutant allele of a Group II mutation is generally observed to be associated with altered immune response capability, which may be manifest as a more vigorous response to infection by pathogenic substances or organisms, increased production of interferon-gamma by CD4 and/or CD8 T cells, an increase in the proportion of T cells of the activated, memory or effector phenotype, reduced susceptibility to viral infection and reduced susceptibility to autoimmune disease.

Accordingly, the inventors have developed screens for identifying individuals who exhibit altered immune responses based on genotyping of Group II mutations, or on screening for altered patterns of CD45 mRNA and protein expression associated with carriage of a Group II mutation. The altered immune response may affect the intensity of the immune response generated in response to exposure of an individual to pathogens. In other conditions, however, the altered immune response may be useful in treating certain diseases, for example autoimmune disorders or the like, in the event that the mutation results in negative regulation and a reduction in the level of response generated.

### SUMMARY OF THE INVENTION

In a first aspect the invention relates to a method of screening a human subject for susceptibility to viral infection and/or pre-disposition to developing severe disease following viral infection, which method comprises screening for the presence or absence in the genome of the subject of one or more polymorphic variants or mutations in the gene encoding CD45 or of one or more polymorphic variants in linkage disequilibrium with or in close physical proximity to a polymorphic locus in the gene encoding CD45.

In one embodiment the mutation in CD45 is characterised in that subjects carrying at least one mutant allele exhibit altered CD45 splicing resulting in a reduction in the proportion of the T cell population carrying the CD45RO splice variant and lacking CD45RA expression (i.e. reduced proportion of CD45RO+ T cells) as compared to subjects not carrying a mutant allele. Such mutations are referred to herein as “Group I” CD45 mutations. In this embodiment subjects having at least one mutant allele are scored as being more susceptible to viral infection and/or more pre-disposed to developing severe disease following viral infection, as compared to subjects who do not carry a mutant allele.

Examples of suitable Group I CD45 mutations include, but are not limited to, C77G, C59A and A54G. Screens can also be carried out using mutations or polymorphic variants in linkage disequilibrium with or close physical proximity to a Group I CD45 mutation.

In a further embodiment the mutation in CD45 is characterised in that subjects carrying at least one mutant allele exhibit altered CD45 splicing resulting in an increase in the proportion of the T cell population carrying the CD45RO splice variant but lacking CD45RA expression (i.e. increased proportion of CD45RO+ T cells) as compared to subjects not carrying a mutant allele. Such mutations are referred to herein as “Group II” CD45 mutations. In this embodiment subjects having at least one mutant allele are scored as being less susceptible to viral infection and/or pre-disposed to developing less severe disease symptoms following viral infection, as compared to subjects who do not carry a mutant allele.

Examples of suitable Group II CD45 mutations include, but are not limited to, A138G. Screens can also be carried out using mutations or polymorphic variants in linkage disequilibrium with or close physical proximity to a Group II CD45 mutation.

In a second aspect the invention relates to a method of screening a human subject for an altered immune response capability, which method comprises screening for the presence or absence in said subject of a mutation in the gene encoding CD45, which mutation is characterised in that subjects carrying at least one mutant allele exhibit altered CD45 splicing resulting in an increase in the proportion of the T cell population carrying the CD45RO splice variant but lacking CD45RA expression, as compared to subjects not carrying a mutant allele (i.e. a Group II CD45 mutation), wherein subjects having at least one mutant allele are scored as having altered immune response capability.

The invention further relates to screens based on analysis of patterns of CD45 mRNA expression or on analysis of CD45 protein isoform expression.

### BRIEF DESCRIPTION OF THE DRAWINGS

FIG. 1 shows a digitized image of a gel and FACs analysis plots. FIG. 1A shows a gel demonstrating detection of Exon A (C77G) polymorphism. The C77G transition introduces a new restriction site for Msp I, which cleaves the mutant PCR product into two fragments of 72 and 83 bp. The presence of an undigested band of 155 bp indicates the presence of the wild type allele. FIG. 1B shows plots illustrating the results of FACS analysis to investigate the pattern of CD45 expression in human peripheral T cells pre- and post-stimulation. PBMC were stimulated with 1 μg/ml PHA and on days 0 and day 10 stained with isoform-specific CD45RO-PE and CD45RA-FITC antibody conjugates and with a CD3-APC antibody conjugate. Analysis was performed on gated CD3+ cells. Panels 1 and 2 show the normal pattern of CD45 expression pre- and post-stimulation: T cell activation is associated with a loss in CD45RA and a gain in expression of CD45RO. Panels 3 and 4 show the pattern of CD45 expression pre- and post-stimulation in a C77G heterozygote: the CD45RA population is largely absent and the T cells remain CD45RA/RO double positive after activation.

FIG. 2 shows a schematic diagram (FIG. 2A) of a family tree indicating the CD45 genotype and phenotype in each member of a family including an individual with HLH (family W). The patient with HLH (5) is indicated by an asterisk. FIG. 2A also shows a digitized image of a gel indicating the identification of the CD45 exon A (C77G) mutation in family W. The C77G transversion introduces a new restriction site for MspI, which cleaves the mutant PCR product into two fragments of 72 and 83 bp (lanes 2, 4, 5 and 6). The presence of an undigested band of 155 bp indicates the presence of the wild type allele in the father and older brother (lanes 1 and 3). FIG. 2B shows plots indicating expression of CD45 isoforms in human peripheral T cells. PBMC were stained with isoform specific CD45RA-FITC and CD45RO-PE together with CD3-APC mAbs. Analysis was performed on gated CD3+ cells. The normal pattern of CD45 isoform expression is characterised by the presence of single CD45RA+ and single CD45RO+ cells. Abnormal CD45 expression was seen in the patient (5), his mother (2) and two siblings (4 and 6). The father (1) and brother (3) have normal CD45 pattern of expression.

FIG. 3 shows a digitized image of a gel and a plot, which illustrate expression of CD45 isoforms in a patient with a common variable immunodeficiency and a history of prolonged faecal excretion of poliovirus. FIG. 3A shows the detection of exon A (C77G) polymorphism. The C77G transition introduces a new restriction site for Msp I, which cleaves the mutant PCR product into two fragments of 72 and 83 bp. The presence of an undigested band of 155 bp indicates the presence of the wild type allele. FIG. 3B shows results of flow cytometric analysis of CD45 splicing in CVID patients. Anti-CD3+ lymphocytes stained with CD45RA-FITC and CD45-RO antibodies are shown. Variant CD45 splicing in the patient with prolonged poliovirus excretion can be identified by the absence of the single CD45RO+ population.

FIG. 4 shows schematic diagrams illustrating the identification of A138G polymorphism in exon 6 of CD45. FIG. 4A shows an A→G transversion in position 138 of exon 6 that was identified. Shown are examples of a common variant homozygote allele, heterozygote and homozygotes. The changed bases are boxed. FIG. 4B provides a schematic structure of exon 6, showing the relative position of the A138G mutation, which is 7 bp from the 3′ end of exon 6. The sequence of exon 6 is shown in a one letter amino acid code and the potential glycosylation sites indicated by arrows. The mutation leads to the amino acid change 47T→A in the coded CD45RC domain. (gcgaacacctca is SEQ ID NO:13), (gcgaacncctca is SEQ ID NO: 14), (gcgaacgcctca is SEQ ID NO:15), (acagcgaacacctcaggtctga is SEQ ID NO:16), (acagcgaacgcctcaggtctga is SEQ ID NO:17), and (TANT/AS is SEQ ID NO:18).

FIG. 5 shows plots illustrating the expression of CD45 isoforms in human peripheral T cells. FIG. 5A shows results when PBMC were stained with isoform specific antibodies against CD45RA and CD45RB or CD45RA and CD45RC together with anti-CD3. Analysis was performed on gated CD3⁺ cells. A138G individuals show a decrease in the proportion of cells expressing both CD45RA and CD45RC or CD45RA and CD45RB isoforms. FIG. 5B shows results of expression of CD45R0, CD45RA. and CD45RC on CD3 cells from A138G and control individuals. PBMC were stained with anti-CD3 together with isoform specific CD45R0 and CD45RA or CD45R0 and CD45RC antibodies. In the 138G variant, an increase in the proportion of CD45R0⁺ cells in seen. Examples are representative of similar analyses of 4 A138G homozygous, heterozygous and control individuals.

FIG. 6 shows a graph and a digitized image of a gel illustrating CD45 RNA expression in PBMC from homozygous G138G and A138A individuals. Total RNA was extracted from unstimulated PBMC. After reverse transcription the resulting cDNA was amplified with primers spanning exons 2-7 of CD45 gene. PBMC from both homozygotes for G138G and common variant A138A allele individuals contained mRNA for the CD45R0 (197 bp), CD45RB (337 bp), CD45RBC (480 bp), CD45RAB (534 bp) and CD45RABC (677 bp) isoforms. FIG. 6A shows densitograms of results when bands in each lane were quantitated and shown on top of the gel corresponding to the respective isoform. The ratio between the intensity of the CD45R0 and CD45RB bands is shown at the right of the gel in FIG. 6B. Data of three representative analyses of 3 G138G homozygotes and three control samples for the common variant A138A allele are shown.

FIG. 7 shows graphs illustrating expression of CD45 isoforms and activation markers on CD4 and CD8 cells from 4 healthy G138G homozygous and 6 A138A homozygous control individuals. FIG. 7A shows the proportions of CD8 and CD4 T cells from G138G and A138A control individuals that are CD45R0+. FIG. 7B shows the proportions of CD8 and CD4. FIG. 7C shows results from T cells that express CDlla^{hi}, CD27, CD28, CD62L, CD95 and CCR7. Means and standard deviations of data expressed as the percentage of CD8 and CD4 T cells from 4 G138G and 6 A138A control individuals are shown.

FIG. 8 shows plots illustrating expression of CD45 isoforms in peripheral T cells in Caucasian HIV seronegative (FIG. 8A) and Ugandan HIV seropositive individuals (FIG. 8B). PBMC were stained with isoform-specific CD45RA-FITC and CD45R0-PE together with CD3-APC mAbs. Analysis was performed on CD3 gated cells. The normal pattern is characterised by the presence of single positive CD45RA+ and CD45R0+ cells. Abnormal expression is seen in the C77G individual with all of the cells expressing CD45RA. In the A54G Ugandan individual there are more double positive CD45RA+R0+ cells compared to the A54A controls.

FIG. 9 shows plots of results from fluorescence activated cell sorter analysis of PBMC from 4 individuals carrying a 77G mutant allele and 4 normal control individuals (C77C homozygotes). T cells were stained for a panel of markers after gating for CD4 (FIG. 9A) and CD8 (FIG. 9B).

### DEFINITIONS

In the context of this application the terms “gene encoding CD45” and “CD45 gene” are used interchangeably and refer to a gene, also referred to as the PTPRC gene, located at gene map locus 1q31-32 (OMIM accession 151460). The complete sequence of the gene is available via publicly accessible genome sequence databases. A list of GenBank accession numbers for individual exons of the gene is provided.

The terms “C77G polymorphism”, “A 138G polymorphism”, “C59A polymorphism” and “A54G polymorphism” may be used herein to refer to the respective polymorphic loci.

When referring to individual alleles of the C77G polymorphism, the terms “mutant allele”, “variant allele”, “C77G variant” and “C77G mutation” should be taken to refer to the 77G allele, i.e. the allele having G at position 77. The terms “normal allele” and “wild type allele” should be taken to refer to the 77C allele, i.e. the allele having C at position 77.

When referring to the carrier status of individual human subjects the term “G77G” refers to an individual homozygous for the 77G allele, the term “C77C” refers to an individual homozygous for the 77C allele and the term “C77G” refers to a heterozygous individual.

The terms “carrier(s) of the 77G allele” and “individual(s) having the 77G variant” refer to both homozygotes for 77G and heterozygotes.

The terms “individual having the C77G variant” or “individual having the C77G mutation” may, depending on the context in which it is used, also refer to any individual having a 77G allele, i.e. encompassing both homozygotes for 77G and heterozygotes.

When referring to individual alleles of the A138G polymorphism, the terms “mutant allele”, “variant allele”, “A138G variant” and “A138G mutation” should be taken to refer to the 138G allele, i.e. the allele having G at position 138. The terms “normal allele” and “wild type allele” should be taken to refer to the 138A allele, i.e. the allele having A at position 138.

When referring to the carrier status of individual human subjects the term “G138G” refers to an individual homozygous for the 138G allele, the term “A138A” refers to an individual homozygous for the 138A allele and the term “A138G” refers to a heterozygous individual.

The terms “carrier(s) of the 138G allele” and “individual(s) having the 138G variant” refer to both homozygotes for 138G and heterozygotes.

The terms “individual having the A138G variant” or “individual having the A138G mutation” may, depending on the context in which it is used, also refer to any individual having a 138G allele, i.e. encompassing both homozygotes for 138G and heterozygotes.

When referring to individual alleles of the C59A polymorphism, the terms “mutant allele”, “variant allele”, “C59A variant” and “C59A mutation” should be taken to refer to the 59A allele, i.e. the allele having A at position 59. The terms “normal allele” and “wild type allele” should be taken to refer to the 59C allele, i.e. the allele having C at position 59.

When referring to individual alleles of the A54G polymorphism, the terms “mutant allele”, “variant allele”, “A54G variant” and “A54G mutation” should be taken to refer to the 54G allele, i.e. the allele having G at position 54. The terms “normal allele” and “wild type allele” should be taken to refer to the 54A allele, i.e. the allele having A at position 54.

The protein encoded by the human CD45 gene exists in multiple isoforms, depending on alternative splicing of exons 4, 5 and 6. “CD45RA” refers to isoforms containing the CD45RA domain encoded by exon 4, “CD54RB” refers to isoforms containing the CD45RB domain encoded by exon 5 and “CD45RC” refers to isoforms containing the CD45RC domain encoded by exon 6, whereas “CD45RO” refers to a low molecular weight isoform which lacks exons 4-6. Where a cell or tissue is referred to herein as “lacking expression” of a particular CD45 isoform this may be taken to mean that substantially no expression of the protein isoform is detectable using standard techniques for analysis of protein expression, for example FACs analysis, Western blotting etc. Where a cell or tissue is referred to herein as “lacking expression” of mRNA encoding a CD45 isoform, this may be taken to mean that substantially no expression of the mRNA is detectable using standard techniques for analysis of mRNA expression, for example RT-PCT, RNase protection, Northern blotting etc.

### DETAILED DESCRIPTION OF THE INVENTION

The invention provides genetic screens based on genotyping of genetic variants or mutations in the CD45 gene for determining susceptibility of human subjects to viral infection and/or for identification of subjects having “altered immune response capability”.

As will be illustrated in the accompanying Example, a mutation (C to G transversion) in the fourth or “A” exon of the CD45 gene has been shown to be associated with HIV-1 infection. In addition, the C77G mutation has been found in a patient with common variable immunodeficiency with persistent viral infection and prolonged excretion of polio virus (this patient was previously described by Misbah et al., Postgrad Med J, 1991, Vol: 67, 301-303; see Example 3) and in a patient infected with EBV (data not shown). Furthermore, the inventors have shown the C77G mutation to be present in patients diagnosed with haemophagocytic lymphohistiocytosis (HLH) (see Example 2). Sporadic cases of HLH are often provoked by viral infection in childhood (Dreyer, et al., Am J Pediatr Hematol Oncol, Vol: 13, 476).

As further illustrated in the examples provided, a mutation (A to G transversion) at position 138 in exon 6 of the CD45 gene, has been shown to be associated with an alteration in cell surface CD45 isoform expression, the transversion resulting in a Threonine to Alanine semi-conservative amino acid change at position 47 of the CD45RC exon 6. The mutation causes dramatic changes in the proportions of T cells expressing CD45 isoforms, with individuals having the 138G variant having an increased proportion of T cells with an activated, memory or effector phenotype, as determined by an increased proportion of CD45RO+ cells, and reduced numbers of cells expressing the CD45 A, B and C isoforms. G138G homozygotes also exhibit altered expression of other leukocyte antigens, namely decreased expression of CD27, CD28, CD62L and CCR7 and increased expression of CD11a and CD95. These changes again indicate that the most prominent effect in 138G individuals is an increase in the proportion of activated/memory T cells having the CD45RO+ phenotype.

Accordingly, as aforesaid, the inventors have concluded that mutations in CD45 can be classified in two groups. Group I CD45 mutations, exemplified by C77G and C59A, are associated with altered splicing of the CD45 mRNA and an altered pattern of CD45 isoform expression on the T cell population, characterized as a reduction in the proportion of T cell population carrying only the CD45RO splice variant (i.e. a reduction in CD45RO+ T cells) in individuals carrying at least one mutant allele, as compared to individuals not carrying a mutant allele. Carriage of a mutant allele of a Group I mutation is generally observed to be associated with increased susceptibility to viral infection and/or a pre-disposition to developing severe disease following viral infection.

Group II CD45 mutations, exemplified by A138G, are associated with altered splicing of the CD45 mRNA and an increase in the proportion of the T cell population having the CD45RO+ phenotype in individuals carrying at least one mutant allele, as compared to individuals not carrying a mutant allele. Carriage of a mutant allele of a Group II mutation is generally observed to be associated with altered immune response capability, which may be manifest as a more vigorous response to infection by pathogenic substances or organisms, increased production of interferon-gamma by CD4 and/or CD8 T cells, an increase in the proportion of T cells of the activated, memory or effector phenotype, reduced susceptibility to viral infection and reduced susceptibility to autoimmune disease.

Genetic Screens Based on Group I Mutations

Genetic screens based on genotyping of one or more Group I CD45 mutations may be used to screen human subjects for susceptibility to viral infection and/or pre-disposition to developing severe disease following viral infection. Individuals having at least one mutant Group I allele are scored as being more susceptible to viral infection and/or pre-disposed to developing severe disease following viral infection.

A “mutant Group I CD45 allele” is defined as a mutant allele of CD45, carriage of which causes (or is associated with) altered splicing of the CD45 mRNA, preventing splicing out of exon 4 of CD45, and an altered pattern of CD45 protein isoform expression manifest as a reduction in the proportion of the T cell population having the CD45RO+ phenotype. Activated/memory T lymphocytes in individuals carrying a Group I mutation continue to express both CD45RA and CD45RO isoforms, in contrast to the “normal” pattern of low molecular weight CD45RO expression (see accompanying examples).

The methods of the invention preferably comprise genotyping of one or more Group I CD45 mutations which have previously been demonstrated to show statistically significant association with susceptibility to viral disease and/or severity of viral disease, for example in a population-based genetic association study or case-controlled study. However, the utility of the invention is not strictly limited to mutations for which a statistically significant disease association has been demonstrated by experiment, since it is possible to predict disease association on the basis of classification as a Group I or Group II mutation.

Suitable Group I mutations include, but are not limited to, C77G, C59A and A54G. Individuals carrying at least one mutant allele of either mutation (i.e. 77G or 59A) are scored as being susceptible to viral infection and/or pre-disposed to developing severe disease following viral infection, as compared to individuals not carrying a mutant allele.

The invention also contemplates screens based on polymorphic variants or mutations (whether or not within the CD45 gene) which have not themselves been shown to be associated with susceptibility to viral infection and/or severity of disease in a population-based study but which are either in linkage disequilibrium with or in close physical proximity to a Group I mutation in CD45.

As would be readily apparent to persons skilled in the art of human genetics, “linkage disequilibrium” occurs between a marker polymorphism (e.g. a DNA polymorphism which is “silent” and a functional polymorphism (i.e. genetic variation which affects phenotype or which contributes to a genetically determined trait) if the marker is situated in close proximity to the functional polymorphism. Due to the close physical proximity, many generations may be required for alleles of the marker polymorphism and the functional polymorphism to be separated by recombination. As a result they will be present together on the same haplotype at higher frequency than expected, even in very distantly related people. As used herein the term “close physical proximity” means that the two markers/alleles in question are close enough for linkage disequilibrium to be likely to arise.

In such screens individuals carrying at least one allele in linkage with a Group I mutant allele will be scored as being susceptible to viral infection and/or pre-disposed to developing severe disease following viral infection.

Genetic Screens Based on Group II Mutations

Genetic screens based on genotyping of one or more Group I CD45 mutations may be used to screen human subjects for “altered immune response capability”. Individuals having at least one Group II mutant allele are scored as having altered immune response capability or altered immunological function as compared to individuals not having a mutant allele.

A “mutant Group II CD45 allele” is defined as a mutant allele of CD45, carriage of which causes (or is associated with) altered splicing of CD45 mRNA which is characterised as a quantitative increase in the level of expression of the CD45RO transcript and an altered pattern of CD45 protein isoform expression manifest as an increase in the proportion of the T cell population having the CD45RO+ phenotype.

Suitable Group II mutations include, but are not limited to, A138G. Individuals carrying at least one mutant allele (i.e. 138G) are scored as having altered immune response capability/altered immunological function, as compared to individuals not carrying a mutant allele.

The invention also contemplates screens based on polymorphic variants or mutations (whether or not within the CD45 gene) which have riot themselves been shown to be associated with susceptibility to viral infection and/or severity of disease in a population-based study but which are either in linkage disequilibrium with or in close physical proximity to a Group II mutation in CD45, e.g. A138G.

In one embodiment the “altered immune response capability” associated with carriage of a Group II mutation such as the 138G allele may be defined as an increase in the proportion of T cells having the activated, memory or effector phenotype, as determined by an increase in the proportion of CD45R0+ T cells, as compared to control individuals homozygous for the “wild type” allele (e.g. 138A). Quantitative analysis of the relative proportions of CD45R0+ positive T cells versus T cells expressing CD45RA, RB or RC isoforms can be carried out using any suitable technique known in the art, such as for example FACS analysis, as illustrated in the accompanying Examples.

T cells having the activated, memory or effector phenotype may be identified on the basis of expression patterns for certain marker proteins. In particular, “activated” T cells may be characterized by decreased expression of CD62L and increased expression of CDlla and CD95, as compared to naive T cells. In individuals carrying Group II CD45 mutations, such as 138G, activated T cells generally express the CD45RO isoform, whereas in individuals carrying Group I mutations, such as 77G, as significant proportion of activated T cells express both CD45RA and CD45RO isoforms. However, in both Group I and Group II mutant carriers “activated” T cells can be identified/characterized on the basis of expression levels of CD62L, CD11a and CD95.

The inventors have further shown that carriage of a Group II CD45 mutation, such as the 138G allele, is associated with increased production of the Th1 cytokine interferon (IFN) gamma by CD4 and CD8 T cells. This provides strong support for the link between carriage of Group II mutations such as the 138G allele and altered immune response capability/altered immune function.

Therefore, in a further embodiment of the invention the “altered immune response capability” associated with carriage of a Group II mutant allele, such as the 138G allele, may be defined as increased production of IFN-gamma by CD4 and/or CD8 T cells, as compared to individuals homozygous for the equivalent wild type allele (e.g. 138A).

The finding that carriage of a Group II mutant allele, such as the 138G allele, is associated with an increase in the proportion of T cells having the activated, memory or effector phenotype and with increased production of the Th1 cytokine IFN-gamma by CD4 and CD8 T cells means that genetic screening of a human subject for carriage of a Group II mutant allele, such as 138G, can provide a useful indication of the immune capability of that subject. Thus, genetic screening for a Group II mutant allele, such as 138G, may potentially be used to evaluate susceptibility of a human subject to (i) any disease wherein disease pathogenesis is affected (either positively or negatively) by the production of an increased proportion of activated, memory or effector T cells, and (ii) any disease wherein disease pathogenesis is affected (either positively or negatively) by increased production of IFN-gamma.

Moreover, genetic screening for a Group II mutant allele, such as 138G, may also potentially be used to evaluate likely severity of disease symptoms for (i) any disease in which the severity of disease symptoms is affected (either positively or negatively) by the production of an increased proportion of activated, memory or effector T cells, and (ii) any disease in which the severity of disease symptoms is affected (either positively or negatively) by increased production of IFN-gamma. The practical applications of screens based on genotyping of Group II mutations such as the A138G polymorphism are therefore potentially very wide within the spectrum of infectious and immune diseases. Susceptibility to disease and/or likely severity of disease will be evaluated based on the presence or absence of the Group II mutant allele (e.g. 138G), depending on whether production of an increased proportion of activated, memory or effector T cells and/or increased production of IFN-gamma is a positive or negative factor from the perspective of the subject under test, i.e. whether these factors promote susceptibility or resistance to the disease in question, or promote severe or mild symptoms of the disease in question.

The increased effector T cell population and/or increased production of the Th1 cytokine IFN-gamma by CD4 and CD8 T cells in individuals having Group II mutations such as the 138G variant may lead to a more vigorous immune response to pathogens. The inventors have shown in the accompanying examples a significant dominant protective effect for the 138G allele in infection with hepatitis B virus (HBV).

Therefore, in a still further embodiment of the invention the “altered immune response capability” associated with carriage of a Group II mutation such as the 138G allele may be defined as a more vigorous response to pathogenic substances or organisms, as compared to individuals homozygous for the wild type allele (e.g. 138A).

Accordingly, genotyping of individuals for a Group II mutation such as the A138G polymorphism may be used to provide an indication of susceptibility to viral infection and/or an indication of the likely severity of disease symptoms following viral infection. In a specific embodiment the viral infection may be infection with hepatitis B virus. In these embodiments, the presence of at least one Group II mutant allele (e.g. 138G) will be taken as an indication of reduced susceptibility to viral infection and/or reduced severity of disease following infection, as compared to individuals homozygous for the wild type allele (e.g. 138A). Such genetic screens might be used, for example, to screen uninfected individuals or those in a very early stage of viral infection in order to evaluate whether the individual is susceptible to viral infection or is pre-disposed, by virtue of their genetic make-up, to develop more or less severe disease symptoms following viral infection, particularly infection with HBV. This knowledge might be useful, for example, in the selection of appropriate treatment (including prophylaxis) for particular individuals. The screens may be of particular use in the screening of neonates and infants in order to determine susceptibility to HBV infection and/or likely severity of disease following infection with HBV.

The above method may be used to provide an indication of susceptibility to viral infection and/or an indication of the likely severity of disease symptoms following viral infection in (i) any viral infection wherein susceptibility to infection and/or the severity of disease symptoms is affected (either positively or negatively) by the production of an increased proportion of activated, memory or effector T cells, and (ii) any viral infection wherein susceptibility to infection and/or the severity of disease symptoms is affected (either positively or negatively) by increased production of IFN-gamma.

IFN-gamma has been shown to be of crucial importance in protective immunity against many infectious diseases, including hepatitis B itself (see Viral pathogenesis Chapter 31 viral hepatitis—Francis V. Chisari and Carlo Ferrari pg 745-778 Editor in chief—Neal Nathanson 1997 Lippincott—Raven publishers, 227 East Washington Square, Philadelphia.Pa. 19106) and tuberculosis. Genetic screens for carriage of the 138G allele can provide a useful indication of disease susceptibility and/or likely disease severity for any infectious disease in which IFN-gamma provides/promotes protective immunity. Other references in the art to the role of IFN-gamma are as follows: Immunobiology: the immune system in health and disease 5th edition Published in 2001 by garland publishing, a member of the Taylor and Francis Group, 29 West 35th Street, New York N.Y.; Charles A. Janeway, Paul Travers, Mark Walport, Mark J. Shlomchik, Szabo S J, Sullivan B M, Peng S L, Glimcher L H. Molecular mechanisms regulating Th1 immune responses. Annu Rev Immunol. 2003;21:713-58. Epub 2001 Dec. 19.; Vandenbroeck K, Goris A Cytokine gene polymorphisms in multifactorial diseases: gateways to novel targets for immunotherapy?. Trends Pharmacol Sci. 2003 June;24(6):284-9.; Adorini L. Cytokine-based immunointervention in the treatment of autoimmune diseases. Clin Exp Immunol. 2003 May;132(2):185-92.; Factor P. Gene therapy for asthma. Mol Ther. 2003 February;7(2):148-52.; Chesler D A, Reiss C S. The role of IFN-gamma in immune responses to viral infections of the central nervous system. Cytokine Growth Factor Rev. 2002 December;13(6):441-54.

In some conditions the increased effector T cell population and/or the increased production of the Th1 cytokine IFN-gamma by CD4 and CD8 T cells in individuals having a Group II mutation such as 138G may be a negative regulator, contributing to the cessation of the immune response, or resulting in a less vigorous or altered immune response, which may be less pathogenic in the individuals who develop the disease or which may reduce the risk of autoimmune disease in carriers of the mutation. The inventors have shown in the accompanying examples a significant dominant protective effect for the 138G allele in the autoimmune disorder Grave's disease and a recessive protective effect for the 138G allele in the autoimmune disease Hashimoto's thyroiditis.

Therefore, in a still further embodiment of the invention the “altered immune response capability” associated with carriage of a Group II mutation such as the 138G allele may be defined as a less vigorous or pathogenic immune response to autoantigen, as compared to individuals homozygous for the wild type allele (e.g. 138A).

Accordingly, genotyping of an individual for a Group II mutation such as the A138G polymorphism may be used to evaluate susceptibility to autoimmune disease and/or as an indicator of the likely severity of autoimmune disease in the individual. In a specific embodiment the autoimmune disease may be Grave's disease or Hashimoto's thyroiditis. In these embodiments, the presence of at least one Group II mutant allele (e.g. 138G) will be taken as an indication of reduced susceptibility to autoimmune disease and/or reduced severity of autoimmune disease symptoms, as compared to individuals homozygous for the equivalent wild type allele (e.g. 138A). Such. genetic screens might be used, for example, to screen asymptomatic individuals thought to be “at risk” of developing autoimmune disease, or individuals manifesting very early symptoms of the disease in order to evaluate whether the individual is pre-disposed, by virtue of their genetic make-up, to develop more or less severe disease symptoms. This knowledge might be useful, for example, in the selection of appropriate treatment (including prophylaxis) for that individual.

An “autoimmune disease” may be defined as a disease in which there is sustained cellular and/or humoral autoreactive immunity and evidence for a pathogenic role of the autoreactive cells or antibodies.

The above method may be used to evaluate susceptibility to autoimmune disease and/or as an indicator of the likely severity of autoimmune disease in (i) any autoimmune disease wherein susceptibility to the disease and/or the severity of disease symptoms is affected (either positively or negatively) by the production of an increased proportion of activated, memory or effector T cells, and (ii) any autoimmune disease wherein susceptibility to the disease and/or the severity of disease symptoms is affected (either positively or negatively) by increased production of IFN-gamma.

The increased effector T cell population and/or increased production of the Th1 cytokine IFN-gamma by CD4 and CD8 T cells in individuals having the 138G variant may still further confer resistance to atopic diseases and allergy. Accordingly, screens for carriage of a Group II mutation such as the 138G allele may further provide useful prognostic information relating to atopic and allergic diseases.

“Allergy” and “atopy” are conditions in which the immune system responds excessively and/or inappropriately to antigens so that tissue damage or other symptoms may result. Immmune responses in these conditions are usually Th2 biased resulting in the production of IgE antibody and in the presence of antigen, the activation of basophils and mast cells and release of histamine and other mediators. Examples of allergy reactions are the acute anaphylactic response of some individuals to bee venom or grass pollens, while dermatitis or asthma represent atopic disorders.

Genotyping of a Group II mutation such as the A138G polymorphism in individuals either “at risk” of developing allergy/atopy or those already manifesting disease symptoms may be useful in selecting appropriate treatment regimes. For example, individuals who develop early disease, e.g. asthma, and are at higher risk because they lack a Group II mutation such as the 138G allele might be scored as candidates for more vigorous early therapy to prevent chronic and more severe disease developing. There is evidence that early BCG immunisation, a Th1 stimulus, is associated with protection against subsequent development of allergy in Japan (Shirakawa T., Enomoto T., Shimazu S. & Hopkins J. M. (1997) The inverse association between tuberculin responses and atopic disorder. Science, 275, 77) so one might predict that Group II mutations such as 138G would be similarly protective.

Genetic screens for carriage of Group II mutations such as 138G may be used diagnostically and/or prognostically, depending on the nature of the disease/condition which it is desired to evaluate, and/or on the status of the patient/subject under test. However, the actual screening methodology will generally be the same regardless of whether the screen is used diagnostically or prognostically. An extremely useful application of the genetic screens is likely to be in predicting the likely outcome of a particular course of treatment/therapy in a given individual, depending on carrier status for the Group II mutation (e.g. 138G). The genetic screens may still further be useful in predicting who will develop disease complications, such as, for example, carcinoma following infection with HBV, or nasopharyngeal carcinoma in EBV infection or adult T cell leukemia or HAM/TSP in HTLV-1 disease.

Genetic screens based on genotyping of Group II mutations such as A138G may also be used in order to predict the likely response of an individual to a vaccine. The “altered immune response capability” associated with carriage of a Group II mutation in a human subjects may affect the ability of an individual to mount an immune response to a challenging antigen or vaccine. Thus, the genetic screens may be used to predict whether vaccination is likely to be successful in a given subject.

In the case of vaccines which induce a protective antibody response, the “altered immune response capability” associated with carriage of the 138G allele will generally pre-dispose to a less strong response, because Th2 cytokines are needed to stimulate production of high antibody titres. Typing for A138G may therefore be used to predict poor vaccine responders who might need an extra boost in order to achieve protection.

In the case of vaccines which induce cellular immunity (Th1 mediated), carriage of 138G will generally pre-dispose to development of a stronger response.

In a particular embodiment the genetic screens may be used to predict the likely response to anti-tumour vaccines. In the case of anti-tumour vaccines that induce a Th1 cellular response, carriers of the 138G allele may be scored as likely to exhibit a more positive response that individuals who do not carry 138G.

General Genotyping Methodology

In the context of the invention, the process of screening for the presence or absence of a mutation or allelic variant in the genome of an individual may advantageously comprise screening for the presence or absence in the genome of the subject of both the common or wild type allele and the variant or mutant allele or may comprise screening for the presence or absence of either individual allele, it generally being possible to draw conclusions about the genotype of an individual at a polymorphic locus having two alternative allelic forms just by screening for one or other of the specific alleles.

The step of screening for the presence or absence of a mutation or allelic variant in the genome of a subject, also referred to herein as “genotyping”, can be carried out using any suitable methodology known in the art and it is to be understood that the invention is in no way limited by the precise technique used to perform such genotyping.

Known techniques for the scoring of single nucleotide polymorphisms include, but are not limited to, mass spectrometry, particularly matrix-assisted laser desorption/ionization time-of-flight mass spectrometry (MALDI-TOF-MS), single nucleotide primer extension and DNA chips or microarrays (see review by Schafer, A. J. and Hawkins, J. R. in Nature Biotechnology, Vol 16, pp33-39 (1998)). The use of DNA chips or microarrays could enable simultaneous genotyping at many different polymorphic loci in a single individual or the simultaneous genotyping of a single polymorphic locus in multiple individuals. SNPs may also be scored by DNA sequencing.

In addition to the above, SNPs are commonly scored using PCR-based techniques, such as PCR-SSP using allele-specific primers (described by Fanning, G. C., et al., Tissue Antigens, 1995; 50: 23-31). This method generally involves performing DNA amplification reactions using genomic DNA as the template and two different primer pairs, the first primer pair comprising an allele-specific primer which under appropriate conditions is capable of hybridising selectively to the wild type allele and a non allele-specific primer which binds to a complementary sequence elsewhere within the gene in question, the second primer pair comprising an allele-specific primer which under appropriate conditions is capable of hybridising selectively to the variant allele and the same non allele-specific primer. Still further PCR-based techniques for scoring SNPs include PCR ELISA and DHPLC.

If the SNP results in the abolition or creation of a restriction site, as is the case with the C77G mutation in the CD45 gene, genotyping can be carried out by performing PCR using non-allele specific primers spanning the polymorphic site and digesting the resultant PCR product using the appropriate restriction enzyme (also known as PCR-RFLP). Restriction fragment length polymorphisms, including those resulting from the presence of a single nucleotide polymorphism, may be scored by digesting genomic DNA with an appropriate enzyme then performing a Southern blot using a labelled probe corresponding to the polymorphic region (see Molecular Cloning: A Laboratory Manual, Sambrook, Fritsch and Maniatis, Cold Spring Harbor Laboratory, Cold Spring Harbor, N.Y.).

The known techniques for scoring polymorphisms are of general applicability and it will be readily apparent to persons skilled in the art that known techniques may be adapted for the scoring of single nucleotide polymorphisms in the CD45 gene.

In the case of the C77G mutation, the preferred technique for genotyping of this single mutation is PCR followed by digestion of the PCR product with the enzyme MspI, as described in the accompanying Example. However, the invention is not intended to be limited to the use of this technique.

In the case of the A138G mutation, the preferred technique for genotyping of this single mutation is ARMS PCR, as illustrated in the accompanying Examples. However, the invention is not intended to be limited to the use of this technique.

Genotyping is preferably carried out in vitro, and is most preferably performed on isolated genomic DNA prepared from a suitable tissue sample obtained from the subject under test. Most commonly, genomic DNA is prepared from a sample of whole blood, according to standard procedures, which are well known in the art.

Most advantageously, it is envisaged that individuals will be simultaneously genotyped for multiple CD45 mutations in order to provide a “profile” of overall immune capability/disease susceptibility. Simultaneous genotyping at multiple loci may be achieved, for example, with the use of “gene chips” or microarrays. It is also contemplated that genotyping of CD45 mutations may be carried out simultaneously with genotyping of polymorphic variants/mutations in genes other than CD45 that are also markers for immune function/disease susceptibility.

Identification and Characterisation of New Mutations

Novel mutations in CD45 may be identified by scanning CD45 genomic sequence for genetic variation. The process of scanning CD45 genomic DNA for the presence of polymorphic variants may be accomplished using any of the techniques known in the art (see review by Schafer and Hawkins, Nature Biotechnology, Vol 16, pp33-39 (1998)). Preferred techniques are listed below:

(a) DNA sequencing: Heterozygous changes appear as two bases at a single position in the sequence. Homozygous variants are found by comparison to a control (i.e. wild-type) sequence.

(b) Heteroduplex analysis: this technique is based on the fact that heteroduplexes exhibit a reduced mobility in non-denaturing polyacrylamide gels compared to homoduplexes. The region to be tested (advantageously around 200 bp) is amplified, denatured and re-natured to itself or control “wild-type” DNA and the duplexes resolved on a non-denaturing gel. The same region of DNA is compared between individuals and differential mobilities indicate sequence differences.

(c) Single-strand conformation polymorphism analysis (SSCP or SSCA): single stranded DNA folds up to form complex structures that are stabilized by weak intramolecular bonds. The electrophoretic mobilities of these structures on non-denaturing polyacrylamide gels is dependent upon chain length and conformation. Typically, PCR amplification products from the region to be tested are heat denatured and rapidly cooled to impede reassociation of complementary strands. The products are then resolved on a non-denaturing gel. The same region of DNA is compared between individuals and differential mobilities indicate sequence differences that exist between the individuals in this region.

(d) Chemical cleavage of mismatches (CCM): a radiolabelled probe is hybridised to the test DNA and mismatches detected by a series of chemical reactions that cleave one strand of the DNA at the site of the mismatch. This sensitive method can be applied to kilobase-length fragments.

(e) Enzymatic cleavage of mismatches: technique similar to CCM, except that the cleavage is performed using an enzyme (e.g. T4 endonuclease VII).

(f) Mass spectrometry: Matrix-assisted laser desorption/ionization time-of-flight mass spectrometry (MALDI-TOF-MS) may be used to compare DNA fragments by sensitive mass determination.

(g) Southern blotting: a labelled probe consisting of a fragment of the linkage region is hybridised to nylon membranes containing genomic DNA from patients and normal controls digested with different restriction enzymes. Large differences in the sizes of the restriction fragments hybridizing with the probe between patients and controls may indicate the presence of a restriction fragment length polymorphism.

(h) Denaturing high performance liquid chromatography (DHPLC): a PCR product is amplified corresponding to the region to be analysed for the presence of mutations. Heteroduplex formation is then analysed through hybridisation following heating and cooling of the PCR products.

The above-listed techniques may be employed to scan CD45 genomic DNA from human subjects in order to identify novel polymorphic variants.

In addition, a significant amount of information regarding known polymorphic variants is to be found in publicly accessible databases such as, for example, the human SNP database accessible via the Website of the Whitehead Institute, Cambridge, Mass., USA. Thus, it is also contemplated to scan/searching of these sources to identify novel mutations/polymorphic variants in CD45 which may be used as the basis of genetic screens.

“New” CD45 mutations and variants may be characterised as Group I or Group II on the basis of their effect on (or association with) CD45 mRNA splicing and CD45 protein isoform expression. Such effects/associations may be investigated by analysing patterns of CD45 mRNA expression in individuals of know genotype, for example using RT-PCR or Northern blotting and/or analysing patterns of CD45 protein isoform expression on T lymphocytes in individuals of known genotype, for example using FACS analysis as described in the accompanying Examples.

Screens Based on Analysis of CD45 mRNA Expression and CD45 Protein Isoform Expression

The invention also relates to screens based on evaluation of the altered patterns of CD45 mRNA or protein isoform expression associated with carriage of CD45 Group I or Group II mutations. These screens are analogous to the genetic screens based on genotyping of CD45 mutations. In essence, all three screening methodologies ultimately provide an indication of carrier status for CD45 mutations, the only differences being whether the screen is carried out at the genomic level or at the level of mRNA or protein expression. Hence, all of the preferred clinical/diagnostic/prognostic uses described for the genetic screens are equally applicable to the screens based on altered mRNA or protein expression.

Accordingly, the invention provides a method of screening a human subject for susceptibility to viral infection and/or pre-disposition to developing severe disease following viral infection which comprises evaluating the pattern of CD45 mRNA expression in the subject, wherein the presence of an abnormal pattern of CD45 mRNA expression characterised by reduced splicing out of exon 4 of the CD45 mRNA and a quantitative decrease in amount of CD45RO transcript is taken as an indication that the subject is susceptible to viral infection and/or pre-disposed to developing severe disease following viral infection.

In one embodiment the abnormal pattern of CD45 mRNA expression is that associated with the presence of a 77G mutant allele of the gene encoding CD45, the presence of this abnormal expression pattern being taken as an indication that the subject is more susceptible to viral infection and/or more pre-disposed to developing severe disease following viral infection, as compared to subjects who do not carry a 77G mutation.

In a further embodiment the abnormal pattern of CD45 mRNA expression is that associated with the presence of a 59A mutant allele of the gene encoding CD45, the presence of this abnormal expression pattern being taken as an indication that the subject is more susceptible to viral infection and/or more pre-disposed to developing severe disease following viral infection, as compared to subjects who do not carry a 59A mutation.

The term “abnormal pattern of CD45 mRNA expression associated with the presence of a 77G mutant allele of the gene encoding CD45” refers to the variant CD45 splicing phenotype described by Thude et al., Eur J Immunol, 1995, Vol: 25(7), 2101-6 and shown to be associated with heterozygosity for the C77G mutation. Individuals homozygous for the C77G mutation are expected to show an exaggeration of the mRNA expression pattern observed in heterozygotes.

The term “abnormal pattern of CD45 mRNA expression associated with the presence of a 59A mutant allele of the gene encoding CD45” refers to the variant CD45 splicing phenotype described by Jacobsen, M. et al., Immunogenetics, 2002, Vol: 54(3), 158-63.

In a further aspect the invention provides a method of screening a human subject for susceptibility to viral infection and/or pre-disposition to developing severe disease following viral infection which comprises evaluating the pattern of CD45 mRNA expression in the subject, wherein the presence of an abnormal pattern of CD45 mRNA expression characterised by a quantitative increase in the level of expression of the CD45RO transcript is taken as an indication that the subject is not susceptible to viral infection and/or is pre-disposed to developing less severe disease following viral infection.

In one embodiment the abnormal pattern of CD45 mRNA expression is that associated with the presence of a 138G mutant allele of the gene encoding CD45, wherein detection of the abnormal pattern of CD45 mRNA expression is taken as an indication that the subject is less susceptible to viral infection and/or pre-disposed to developing less severe disease following viral infection, as compared to subjects who do not carry a 138G mutant allele.

The invention also relates to a method of screening a human subject for an altered immune response capability, which method comprises evaluating the pattern of CD45 mRNA expression in said individual. The presence of an abnormal pattern of CD45 mRNA expression characterised by a quantitative increase in the level of expression of the CD45RO transcript is taken as an indication that the subject has an altered immune response capability.

Again, in one embodiment the abnormal pattern of CD45 mRNA expression is that associated with the presence of a 138G mutant allele of the gene encoding CD45. Detection of the abnormal pattern of CD45 mRNA expression is taken as an indication that the subject has an altered immune response capability, as compared to subjects who do not carry a 138G mutant allele.

The “abnormal pattern of CD45 mRNA expression associated with the presence of a 138G mutant allele of the gene encoding CD45” refers to the variant pattern of CD45 mRNA expression described by Stanton et al., PNAS, 2003, Vol 100(10), 5997-6002, the contents of which are incorporated herein by reference.

The screens based on analysis of patterns of CD45 mRNA expression are preferably carried out in vitro, for example by analysis of preparations of total or mRNA isolated from a tissue or cell type which expresses CD45 (e.g. peripheral blood lymphocytes). Suitable RNA analysis techniques which may be used to determine the pattern of CD45 mRNA expression in accordance with the invention include, but are not limited to, RT-PCR, NASBA, Northern blotting and RNAse protection assays, starting from a sample of total or mRNA prepared from a tissue which expresses CD45 (e.g. PBLs). It is most preferred to use a technique which permits quantitative analysis of mRNA expression, an in particular a technique that allows quantitation of the levels of expression of at least CD45RO and CD45RA transcripts, and most preferably which allows quantitation of the levels of expression of all CD45 splice variants.

The invention also relates to analogous screening methods based on analysis of CD45 protein isoform expression.

Accordingly, the invention provides a method of screening a human subject for susceptibility to viral infection and/or pre-disposition to developing severe disease following viral infection which comprises evaluating the pattern of CD45 protein expression in the subject, wherein the presence of an abnormal pattern of CD45 protein expression characterised as a reduction in the proportion of T lymphocytes expressing the CD45RO isoform but not expressing CD45RA is taken as an indication that the subject is susceptible to viral infection and/or pre-disposed to developing severe disease following viral infection.

In one embodiment the abnormal pattern of CD45 protein expression is that associated with the presence of a 77G mutant allele of the gene encoding CD45. The presence of the abnormal pattern of CD45 protein expression is taken as an indication that the subject is more susceptible to viral infection and/or more pre-disposed to developing severe disease following viral infection, as compared to subjects who do not carry a 77G mutant allele.

In another embodiment the abnormal pattern of CD45 protein expression is that associated with the presence of a 59A mutant allele of the gene encoding CD45. The presence of the abnormal pattern of CD45 protein expression is taken as an indication that the subject is more susceptible to viral infection and/or more pre-disposed to developing severe disease following viral infection, as compared to subjects who do not carry a 59A mutant allele.

The invention also provides method of screening a human subject for susceptibility to viral infection and/or pre-disposition to developing severe disease following viral infection which comprises evaluating the pattern of CD45 protein expression in the subject, wherein the presence of an abnormal pattern of CD45 protein expression characterised by an increase in the proportion of T lymphocytes expressing the CD45RO isoform but not CD45RA is taken as an indication that the subject is not susceptible to viral infection and/or is pre-disposed to developing less severe disease following viral infection.

In one embodiment the abnormal pattern of CD45 protein expression is that associated with the presence of a 138G mutant allele of the gene encoding CD45. The presence of the abnormal pattern of CD45 protein expression is taken as an indication that the subject is less susceptible to viral infection and/or pre-disposed to developing less severe disease following viral infection, as compared to subjects who do not carry a 138G mutant allele.

The invention still further relates to a method of screening a human subject for an altered immune response capability, which method comprises evaluating the pattern of CD45 protein expression in said individual, wherein the presence of an abnormal pattern of CD45 protein expression characterized as an increased in the proportion of T lymphocytes expressing the CD45RO isoform but not CD45RA is taken as an indication that the individual has an altered immune response profile as compared to individuals that do not carry said mutation.

Again, in one embodiment the abnormal pattern of CD45 protein expression is that associated with the presence of a 138G mutant allele of the gene encoding CD45, wherein detection of the abnormal pattern of CD45 protein expression is taken as an indication that the subject has an altered immune response capability, as compared to subjects who do not carry a 138G mutant allele.

The term “pattern of CD45 protein expression associated with the presence of a 77G mutant allele of the gene encoding CD45” refers to the variant pattern of expression of CD45 protein isoforms on peripheral T cells shown to be associated with heterozygosity for the C77G mutation, as described by Thude et al., Eur J Immunol, 1995, Vol: 25(7), 2101-6, the contents of which are incorporated herein by reference. The normal pattern of CD45 protein expression is characterised by loss of expression of the CD45RA isoform and gain in expression of CD45RO after T cell activation. Individuals heterozygous for C77G are characterised continuous expression of the CD45RA isoform on activated and memory T cells, i.e. the T cells remain CD45RA/RO double positive after activation. Individuals homozygous for the C77G mutation are expected to show very little expression of CD45RO at the cell surface.

The term “pattern of CD45 protein expression associated with the presence of a 59 A mutant allele of the gene encoding CD45” refers to the variant pattern of expression of CD45 protein isoforms on peripheral T cells described by Jacobsen, M. et al., 2002, Immunogenetics, Vol: 54(3), 158-163, the contents of which are incorporated herein by reference.

The term “pattern of CD45 protein expression associated with the presence of a 138G mutant allele of the gene encoding CD45” refers to the variant pattern of expression of CD45 protein isoforms on peripheral T cells described by Stanton et al., PNAS, 2003, Vol 100(10), 5997-6002.

Analysis of the CD45 protein isoform expression pattern on peripheral T cells is preferably carried out using flow cytometry, as described in the accompanying example. Individuals heterozygous for C77G are characterised by the absence of a CD45RA negative population of leucocytes. Further suitable techniques which may be used to assess the pattern of expression of CD45 isoforms include immunoprecipitation and Western blotting.

In a further aspect, the invention provides a method of screening an individual for an altered immune response, which method comprises evaluating the pattern of CD45 protein expression in said individual, wherein the presence of an abnormal pattern of CD45 protein expression associated with the presence of a 138G mutant allele of the gene encoding CD45 is taken as an indication that the individual has an altered immune response profile as compared to individuals that do not carry said mutation.

The term “pattern of CD45 protein expression associated with the presence of a A138G mutant allele of the gene encoding CD45” refers to the variant pattern of expression of CD45 protein isoforms on peripheral T cells shown to be associated with heterozygosity for the A138G mutation. The normal pattern of CD45 protein expression is characterised by loss of expression of the CD45RA isoform and gain in expression of CD45RO after T cell activation.

The screens based on analysis of the CD45 protein isoform expression pattern on peripheral T cells are preferably carried out in vitro on samples removed from the subject under test. Analysis of the CD45 protein isoform expression pattern on peripheral T cells is preferably carried out using flow cytometry, as described in the accompanying examples, but other techniques for the analysis of protein expression may be used. Further suitable techniques which may be used to assess the pattern of expression of CD45 isoforms include immunoprecipitation and Western blotting.

Generally it is preferred to analyse CD45 protein isoform expression using a technique which permits quantitation of the levels of expression of at least the CD45RO and CD45RA isoforms, and more preferably using a technique that allows quantitation of the levels of expression of all CD45 protein isoforms.

The screening methods of the invention may be used to identify human subjects who are susceptible or pre-disposed to viral infection by virtue of their genetic make-up. This may allow intervention with preventative therapies aimed at boosting immune function. Screening for increased susceptibility to viral infections and/or for risk of developing more severe virus-induced disease would be important for individuals at increased risk of life threatening virus infections. These may include, for example, gay men and intravenous drug users or medical personnel working in renal dialysis units. “At risk” individuals may be counselled or excluded from high risk situations and measures may be taken to ensure that vaccination results in protective antibody titres in these individuals where a vaccine is available. Screening may also be useful for predicting whether individuals with chronic viral infection, such as for example Hepatitis B or C, are likely to be refractory to expensive immunotherapy.

Kits

The invention also relates to kits for use in carrying out the methods of the invention.

In particular embodiments the invention provides:

(1) A kit for use in genotyping individuals for the A138G polymorphism by amplification refractory mutation system (ARMS) PCR, the kit comprising the following oligonucleotide primers:

| | |
|---|---|
| 5′-GGAGAAGTGCTTGAAGATT-3′, | (SEQ ID NO:1) |
| 5′-CGTATCAGTCTGGACTCCA-3′, | (SEQ ID NO:2) |
| and | |
| 5′-CGTATCAGTCTGGACTCCG-3′. | (SEQ ID NO:3) |

(2) A kit for use in genotyping individuals for the C77G polymorphism by PCR RFLP, the kit comprising the following oligonucleotide primers:

5′-GACTACAGCAAAGATGCCCAGTG-3′ (SEQ ID NO:4) and

5′-GGGATACTTGGGTGGAAGTA-3′ (SEQ ID NO:5), optionally with a supply of the restriction enzyme Msp I.

(3) A kit for use in genotyping individuals for the C77G polymorphism by amplification refractory mutation system (ARMS) PCR, the kit comprising the following oligonucleotide primers:

| | |
|---|---|
| 5′-CATATTTATTTTGTCCTTCTCCCA-3′, | (SEQ ID NO:6) |
| 5′-GAAAGTTTCCACCAACGG-3′ | (SEQ ID NO:7) |
| and | |
| 5′-GAAAGTTTCCACGAACGC-3′. | (SEQ ID NO:8) |

(4) A kit for use in genotyping individuals for the A54G polymorphism and/or the C77G polymorphism by DHPLC, the kit comprising the following oligonucleotide primers:

| | |
|---|---|
| 5′-CATATTTATTTTGTCCTTCTCCCA-3′ | (SEQ ID NO:9) |
| and | |
| 5′-GTGCAGAAATGCAGGAAAT-3′. | (SEQ ID NO:10) |

(5) A kit for use in genotyping individuals for the A138G polymorphism by DHPLC, the kit comprising the following oligonucleotide primers:

| | |
|---|---|
| 5′-GGAGAAGTGCTTGAAGATT-3′ | (SEQ ID NO:1) |
| and | |
| 5′-GTGCCAGATATTATTTGTAGG-3′. | (SEQ ID NO:10) |

Oligonucleotide primers may be supplied in the kits ready for use, as concentrates requiring dilution before use, or in a lyophilised or dried form requiring reconstitution prior to use. If required, the kits may further include a supply of a suitable diluent for dilution or reconstitution of the primers. Optionally, the kits may further comprise supplies of reagents, buffers, enzymes etc for use in carrying out PCR amplification. The preferred features of such reagents are described in the Materials and Methods sections of the accompanying Examples.

The invention will be further understood with reference to the following, non-limiting, Experimental Examples:

### EXAMPLES

### Example 1—Association Between C77G and HIV Infection

Genomic DNA samples and cryopreserved PBMC were obtained from 182 HIV-1 infected patients enrolled at the St Stephen's Clinic, Chelsea and Westminster Hospital, as a part of a functional immunological study. An additional 15 DNA samples from individuals identified as HIV-1-infected at seroconversion, were supplied by Dr P. Borrow. Ethical approval was obtained and the patients gave written consent. The control group consisted of 236 healthy volunteer blood donors, obtained through the local blood bank of the UK National Blood Transfusion Service.

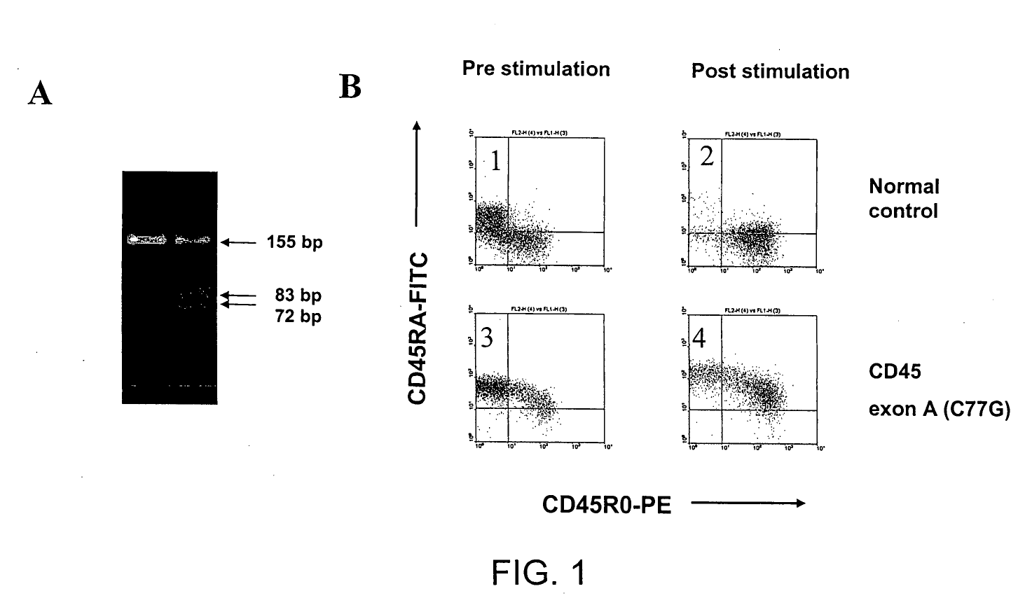

The detection of exon A (C77G) was performed on genomic DNA amplified by PCR using forward (5′-GACTACAGCAAAGATGCCCAGTG-3′ (SEQ ID NO:4)) and reverse primers (5′-GGGATACTTGGGTGGAAGTA-3′ (SEQ ID NO:5)). The C77G transition introduces a new restriction site for Msp I, which cleaves the mutant PCR product into two fragments of 72 and 83 bp. The presence of an undigested band of 155 bp indicates the presence of the wild type allele (illustrated in FIG. 1A).

The presence of the CD45 exon A mutant allele was confirmed by sequencing and flow cytometric analysis on C77G positive samples. PBMC were stimulated with PHA and on days 0 and day 10 stained with isoform specific CD45RO-PE and CD45RA-FITC antibody conjugates (obtained from Dako and Sigma, respectively) together with CD3-APC antibodies (obtained from Pharmingen). Analysis was performed on gated CD3+ T cells. The normal pattern of CD45 splicing is characterised by loss of CD45RA and gain in expression of CD45RO associated with the activated/memory function (A and B, FIG. 1). Variant CD45 splicing can be identified by the absence of the single CD45RO+ population and even after 10 days of stimulation the T cells remain CD45RA/RO double positive (C and D, FIG. 1).

Using PCR and Msp I digestion analysis 11 individuals with the exon A (C77G) mutation were identified out of 197 HIV-1 patients and 4 out of 236 healthy donors (Table 1). The presence of the C77G mutation in these individuals was confirmed by flow cytometric analysis of CD45 protein expression. Using two-tailed Fisher's exact test to test for the association between C77G mutation and HIV-1 infection, a statistically significant association was demonstrated (p=0.03).

The results of this study clearly indicate that exon A (C77G) transversion and abnormal CD45 splicing are associated with HIV-1 infection.

TABLE 1
Frequency of CD45 exon A (C77G) mutation in
HIV patients and healthy controls.

| | | Number with | |
|---|---|---|---|
| | Total number | Exon A (C77) | Frequency |
| HIV | 197 | 11 | 5.6% |
| Healthy donors | 236 | 4 | 1.7% |

### Example 2—Abnormal CD45 Splicing in Haemophagocytic Lymphohistiocytosis

Two patients with a similar defect in CD45 splicing associated with familial erythrophagocytic lymphohistiocytosis (Bujan, W., L. Schandene, A. Ferster, C. De Valck, M. Goldman, and E. Sariban. 1993. Lancet 342:1296) and haemophagocytic lymphohistiocytosis (Wagner, R., G. Morgan, and S. Strobel. 1995. Clin. Exp. Immunol. 99:216.) have been previously described. Haemophagocytic lymphohistiocytosis (HLH) is a rare disorder characterised by disregulated activation of T lymphocytes and macrophages (Arico, M., S. Imashuku, R. Clementi, S. Hibi, T. Teramura, C. Danesino, D. A. Haber, and K. E. Nichols. 2001. Blood 97:1131). HLH is genetically heterogenous with both familial and sporadic forms described (Janka, G. E. 1983. Eur. J. Pediatr. 140:221; Dreyer, Z. E., B. L. Dowell, H. Chen, E. Hawkins, and K. L. McClain. 1991. Am J Pediatr Hematol Oncol vol. 13:476; Dufourcq-Lagelouse, R., N. Jabado, F. Le Deist, J. L. Stephan, G. Souillet, M. Bruin, E. Vilmer, M. Schneider, G. Janka, A. Fischer, and G. de Saint Basile. 1999. Am. J. Hum. Genet. 64:172).

Because of the similarity of the abnormal CD45 splicing in the two previously described HLH patients, to variant CD45 splicing in apparently normal individuals, we investigated the association of the known C77G mutation and HLH syndrome.

Materials and Methods

Materials

Fresh blood was obtained from the previously described family W. (Wagner, R., G. Morgan, and S. Strobel. 1995. Clin. Exp. Immunol. 99:216) and family G. (with two children with HLH) from the Immunobiology Unit, Institute for Child Health, London, UK. PBMC were isolated by centrifugation on a Ficoll-Paque (Amersham Pharmacia Biotech, Buckinghamshire, UK) density gradient and genomic DNA was extracted by standard procedures (Sambrook, J., E. F. Fritsch, and T. Maniatis. 1989. Molecular Cloning: A Laboratory Manual. Cold Spring Harbour Laboratory Press). Genomic DNA samples from family R. (Bujan, W., L. Schandene, A. Ferster, C. De Valck, M. Goldman, and E. Sariban. 1993. Lancet 342:1296.) together with genomic DNA samples from 19 unrelated HLH patients were provided by the Universita di Pavia, Italy.

Genotyping for CD45 Exon A (C77G) Mutation

Genomic DNA was amplified by PCR using forward (5′-GACTACAGCAAAGATGCCCAGTG-3′ (SEQ ID NO:4)) and reverse (5′-GGGATACTTGGGTGGAAGTA-3′ (SEQ ID NO:5)) primers as previously described (Tchilian, E. Z., D. L. Wallace, N. Imami, H. X. Liao, C. Burton, F. Gotch, J. Martinson, B. F. Haynes, and P. C. Beverley. 2001. J. Immunol. 166:6144.). The C77G transversion introduces a new restriction site for MspI (Amersham Pharmacia Biotech), which produces two additional fragments of 72 bp and 83 bp upon digestion in the mutant allele. The PCR and digestion products were analysed on VisiGel Separation Matrix (Stratagene, La Jolla, Calif.).

Flow Cytometric Analysis

Flow cytometric analysis of CD45 variant splicing was performed as previously described (Tchilian, E. Z., D. L. Wallace, N. Imami, H. X. Liao, C. Burton, F. Gotch, J. Martinson, B. F. Haynes, and P. C. Beverley. 2001. J. Immunol. 166:6144.). Briefly, 2×10⁵ PBMC were stained with APC-conjugated CD3 (Pharmingen, SanDiego, Calif.) along with FITC-conjugated CD45RA (Sigma, St Louis, Mo.) and PE-conjugated CD45RO (Dako, Glostrup, Denmark) mAbs in a single step at 4° C. for 20 minutes and washed with PBS, containing 0.5% BSA. Isotype matched mAbs were used as controls. 10,000 events per sample were collected on FACSCalibur (Becton Dickenson, Mountain View, Calif.) and analysed with Cellquest software.

Results

CD45 Exon A (C77G) Mutation is the Cause of CD45 Abnormal Splicing in Two Families with HLH

Material was obtained from two patients with HLH, previously described as exhibiting CD45 abnormal splicing as characterised by the lack of the single CD45RO+ T cell population (Bujan, W., L. Schandene, A. Ferster, C. De Valck, M. Goldman, and E. Sariban. 1993. Lancet 342:1296; Wagner, R., G. Morgan, and S. Strobel. 1995. Clin. Exp. Immunol. 99:216). Subsequently a C77G mutation in exon A of CD45 has been shown to be responsible for the abnormal CD45 splicing in T lymphocytes (Thude, H., J. Hundrieser, K. Wonigeit, and R. Schwinzer. 1995. Eur. J. Immunol. 25:2101; Zilch, C. F., A. M. Walker, M. Timon, L. K. Goff, D. L. Wallace, and P. C. Beverley. 1998. Eur. J. Immunol. 28:22). We therefore genotyped these patients and members of their families for the presence of the CD45 exon A C77G mutation.

Patient W. was the third child of healthy unrelated British Caucasian parents (Wagner, R., G. Morgan, and S. Strobel. 1995. Clin. Exp. Immunol. 99:216.). He presented aged 3 mo with fever, diarrhoea, pallor, increasing irritability and marked cervical lymphoadenopathy and hepatosplenomegaly. Laboratory investigations revealed pancytopenia, coagulopathy and hypertryglyceridemia. The diagnosis of HLH was made from the bone marrow aspirate, which showed haemophagocytosis. There was a good response to initial treatment with dexamethasone and etoposide and he underwent allogeneic bone marrow transplantation from his HLA identical brother.

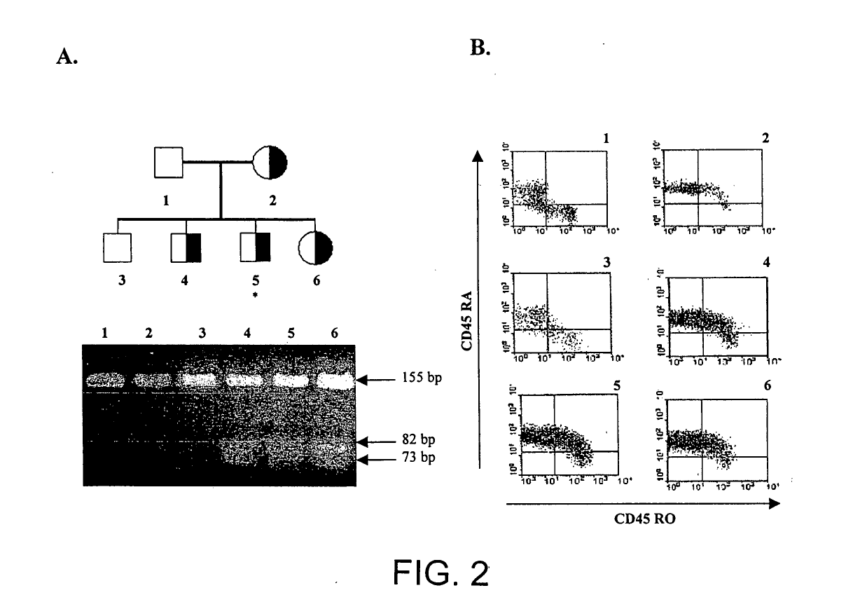

Using PCR and MspI restriction analysis we found that patient W. his mother and two siblings were heterozygous for the mutant C77G allele while the father and the oldest brother had wild type CD45 (FIG. 2A). These results were confirmed by flow cytometric analysis on PBMC from family W. (FIG. 2B). All of the family members genotyped as having the C77G mutation exhibit phenotypically abnormal CD45 splicing. (These results are in agreement with the initial report of family W. that at that time had only three children.)

Patient R. was a first child of consanguineous Belgian Caucasian parents (Bujan, W., L. Schandene, A. Ferster, C. De Valck, M. Goldman, and E. Sariban. 1993. Lancet 342:1296.). The patient presented at the age of 2 mo with fever, hepatosplenomegaly, neutropenia, thrombocytopenia, hypofibrinogenemia and hypertriglyceridemia. He responded to initial treatment with etoposide and underwent bone marrow transplantation from his haploidentical half-sibling and remained asymptomatic over 8 years later. Two older siblings from a previous marriage died during infancy of a histiocytic disorder. Genotyping for the C77G polymorphism revealed that the patient and his mother are heterozygotes while his father and grandmother (also the father's sister) carried wild type CD45 (data not shown). Taken together these results show that the CD45 exon A (C77G) mutation is the cause for the CD45 abnormal splicing in the two HLH patients.

Analysis of CD45 Exon A C77G Mutation in 21 HLH Patients

Since two families with HLH were identified with abnormal CD45 splicing and the C77G mutation we next investigated the pattern of CD45 expression in other HLH patients. Using PCR and MspI restriction analysis we genotyped 21 patients with HLH (including the two affected sibs from family G.) for the presence of CD45 exon A (C77G) mutation. We did not find the mutant C77G allele in any of these patients.

Although taken together the above results show a frequency of 1:10 in HLH type 2 (with identified mutations in the PRF1 gene), or 2:23 for HLH overall, the number of subjects included in the study was very small and it is therefore impossible to draw statistically significant conclusions. Extensive studies on the frequency of C77G have not been performed but we have shown the frequency of the C77G individuals to be about 1.76% in the UK (Tchilian, E. Z., D. L. Wallace, N. Imami, H. X. Liao, C. Burton, F. Gotch, J. Martinson, B. F. Haynes, and P. C. Beverley. 2001. J. Immunol. 166:6144.), while in Germany the frequency has been reported to be less then 1% and in North America to be higher (3.6 %) (Jacobsen, M., D. Schweer, A. Ziegler, R. Gaber, S. Schock, R. Schwinzer, K. Wonigeit, R. B. Lindert, O. Kantarci, J. Schaefer-Klein, H. I. Schipper, W. H. Oertel, F. Heidenreich, B. G. Weinshenker, N. Sommer, and B. Hemmer. 2000. Nat. Genet. 26:495.).

### Example 3—Abnormal CD45 Splicing in a Patient With a Common Variable Immunodeficiency and a History of Prolonged Faecal Excretion of Poliovirus

Common variable immunodeficiency (CVID) is an acquired primary antibody deficiency characterised by recurrent encapsulated bacterial infection and autoimmune disease. The underlying pathogenic defects are heterogeneous with at least four groups of patients being identified according to their ability to secrete immunoglobulin in vitro (Bryant A, Calver N C, Toubi E, Webster A D, Farrant J. Clin Immunol Immunopathol 1990; 56: 239-48), presence of granulomatous disease and autoimmune disease. In general, patients with CVID are not prone to viral infections though infection with enteroviruses may be a potential problem (Rudge P, Webster A D, Revesz T, et al. Brain 1996; 119: 1-15). In view of the possibility that abnormalities in CD45 splicing might contribute to impaired anti-viral responses we report here on a patient with CVID and a history of prolonged poliovirus excretion (Misbah S A, Lawrence P A, Kurtz J B, Chapel H M. Postgrad Med J 1991; 67: 301-303), who exhibited abnormal CD45 splicing caused by the C77G polymorphism.

Materials and Methods

Case History

The patient was a 49 year old Caucasian male with CVID who had prolonged faecal excretion of a non-vaccine strain type II poliovirus between January 1987 and July 1988. In view of his occupation as a nurse and the attendant occupational health implications of prolonged poliovirus excretion, his case history was previously reported (Misbah S A, Lawrence P A, Kurtz J B, Chapel H M. Postgrad Med J 1991; 67-301-303). In brief CVID was diagnosed at the age of 18 years when he presented with hypogammaglobulinaemia (IgG2.8 g/l (ref. range 8-16), IgA 0.48 g/l (ref. range 1.4-4.2), IgM undetectable (ref. range 0.5-2.0)) on a background of delayed puberty, intermittent diarrhoea and intestinal nodular lymphoid hyperplasia. He was lost to follow-up between 1972 and 1986. Although he did not suffer from recurrent infections, it was thought prudent to commence him on intra-muscular immunoglobulin therapy in January 1987 because of his occupation as a nurse. He has been maintained on replacement immunoglobulin since, switching from intra-muscular to subcutaneous immunoglobulin in September 1998. Trough serum IgG levels have varied between 4.4 to 6.1 g/l over the past 2 years. His clinical progress on immunoglobulin replacement has been excellent with only occasional episodes of diarrhoea.

Materials

Fresh EDTA blood was obtained from the patient (Misbah S A, Lawrence P A, Kurtz J B, Chapel H M. Postgrad Med J 1991; 67: 301-303) via the Department of Immunology, John Radcliffe Hospital, Oxford, UK. Genomic DNA was extracted by standard procedure (Sambrook J, E. F. Fritsch, and T. Maniatis. Molecular Cloning: A Laboratory Manual. Cold Spring Harbour Laboratory Press 1989) and monoclonal antibody staining performed as described below.

Genotyping for CD45 Exon A (C77G) Mutation

Genomic DNA was amplified by PCR using forward (5′-GACTACAGCAAAGATGCCCAGTG-3′ (SEQ ID NO:4)) and reverse (5′-GGGATACTTGGGTGGAAGTA-3′ (SEQ ID NO:5)) primers as previously described (Tchilian E Z, Wallace D L, Imami N, et al. J Immunol 2001; 166: 6144-8). The C77G transversion introduces a new restriction site for MspI (Amersham Pharmacia Biotech), which produces two additional fragments of 72 bp and 83 bp upon digestion in the mutant allele. The PCR and digestion products were analysed on a VisiGel Separation Matrix (Stratagene, La Jolla, Calif.).

Flow Cytometric Analysis

Flow cytometric analysis of CD45 variant splicing was adapted from the method previously described (Tchilian E Z, Wallace D L, Imami N, et al. J Immunol 2001; 166: 6144-8). Briefly, 50 μl of EDTA blood was stained with PerCP-conjugated CD3, FITC-conjugated CD45RA and PE-conjugated CD45RO (Becton Dickinson, Oxford, UK) monoclonal antibodies for 15 minutes in the dark at room temperature. Red blood cells were lysed by addition of 1 ml of FACSlyse (Becton Dickinson) for 10 minutes. Lysed stained cells were washed twice with sheath fluid (Becton Dickinson) before being fixed in 0.4 ml of 1% paraformaldehyde and analysed on a FACScan flow cytometer (Becton Dickinson) using Cellquest software. 10,000 events per sample were collected and isotype matched mabs were used as controls.

Results

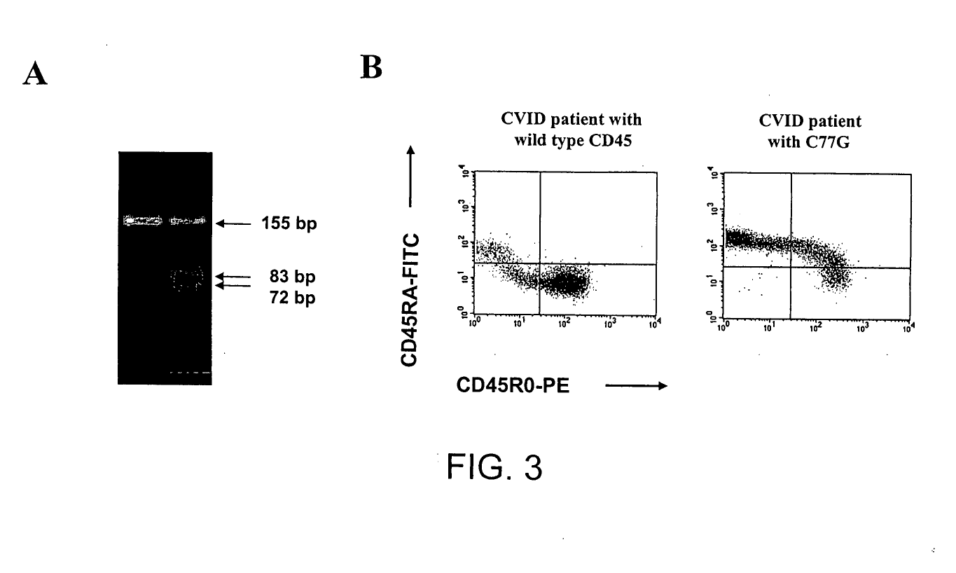

Using PCR and MspI restriction analysis we found that the patient was heterozygous for the mutant C77G allele (FIG. 3A). This result was confirmed by flow cytometric analysis on PBMC from the patient. As shown on FIG. 3B the variant pattern of CD45 splicing can be identified by the absence of the single CD45RO+ T cell population. Taken together these results show that the patient exhibits abnormal CD45 splicing caused by the C77G polymorphism in the gene encoding CD45.

### Example 4—Identification of A138G Polymorphism

Materials and Methods

Materials

175 Japanese genomic DNAs were collected from Osaka City University Medical School, Japan (49 of which were from patients with malignant gynaecological cancer). Peripheral blood mononuclear cells (PBMC) were isolated by centrifugation on a Ficoll-Paque (Amersham Pharmacia Biotech, Buckinghamshire, UK) density gradient and genomic DNA was extracted using DNA blood Minikit (Qiagen K. K., Tokyo, Japan). 209 Ugandan samples were provided by J. Whitworth and A. Hill (Wellcome Trust Centre for Human Genetics, Oxford, UK) (Tchilian, E. Z. et al., Immunogenetics 53: 980-983 (2002). 181 genomic DNA from British individuals consisted of 96 samples obtained through the local Blood Bank of the UK National Blood Transfusion Service, London, UK and 85 provided by Cancer & Immunogenetics Laboratory (Cancer Research UK, Oxford, UK). 72 Orkney samples were provided by Cancer & Immunogenetics Laboratory (Cancer Research UK, Oxford, UK), 48 Korean samples by J. C. Kim (College of Medicine and Asan Medial Centre, University of Uslan, Seoul), 74 Russian and 65 Tatar samples by Russian Rusibakiev (Academy of Science, Tashkent, Uzbekistan). Ethical approval was obtained and the patients gave consent for the study.

Denaturing High Performance Liquid Chromatography (DHPLC) and Sequencing

Genomic DNA was amplified by PCR using the following primers flanking the relevant exons: ex4 forward (5′-CATATTTATTTTGTCCTTCTCCCA-3′ (SEQ ID NO:6)) and ex4 reverse (5′-GTGCAGAAATGCAGGAAAT-3′ (SEQ ID NO:9)), ex6 forward (5′-GGAGAAGTGCTTGAAGATT-3′ (SEQ ID NO:1)) and ex6 reverse (5′-GTGCCAGATATTATTTGTAGG-3′(SEQ ID NO:10)), generating fragments of 384 and 372 bps respectively. A two stage 34 cycle PCR was performed which included an initial 10 min denaturation at 95° C., then 14 cycles of 30 s at 95° C., 30 s at 61.5° C., 30 s at 72° C., followed by 20 cycles of 30 s at 95° C., 30 s annealing at 54° C. for exon 4 and 58° C. for exon 6, 30s at 72° C., and a final 6 min extension at 72° C. PCR reactions were performed in a volume of 50 μl, containing 10 pmol of each primer, 200 μM dNTP, 2.5 mM MgCl₂ and 0.5 U of Amplitaq Gold (Perkin Elmer Life Sciences, Boston, Mass.) in 1×KCL Perkin Elmer buffer II. PCR products were resolved on 2% agarose, then hybridised for 4 min at 95° C., followed by 42 cycles of 1 min at 95° C. dropping by 1.6° C./cycle. Products were run on the DHPLC machine (Transgenomic WAVE, Transgenomic Ltd, Crewe, UK). Purified PCR products were subjected to automated sequencing using the same primers as for DHPLC.

Amplification Refractory Mutation System (ARMS) PCR

To detect carriers of the exon 4 C77G and exon 6 A138G mutations, we used amplification refractory mutation system (ARMS) PCR, with two separate reaction. mixes, containing one forward primer and one of the two reverse primers. For exon 4 the original forward primer was used (5′-CATATTTATTTTGTCCTTCTCCCA-3′ (SEQ ID NO:6)) amplifying both the wild type and the variant allele. The reverse primer ex4 rev A (5′-GAAAGTTTCCACCAACGG-3′ (SEQ ID NO:7)) amplified only the wild type allele, while ex4 rev B (5′-GAAAGTTTCCACGAACGC-3′ (SEQ ID NO:8)) amplified only the variant allele. Similarly for exon 6 the original forward primer was used (5′-GGAGAAGTGCTTGAAGATT-3′ (SEQ ID NO:1)) and ex6 rev A (5′-CGTATCAGTCTGGACTCCA-3′ (SEQ ID NO:2)) to amplify the wild type or ex6 B (5′-CGTATCAGTCTGGACTCCG-3′ (SEQ ID NO:3)), amplifying the mutant allele only. Annealing temperatures were 56° C. for C77G and 60.5° C. for A138G. ARMS PCR products were resolved by Alkaline-Mediated Differential integration (AMDI) (Bartlett, S., Straub, J., Tonks, S., Wells, R. S., Bodmer, J. G. & Bodmer, W. F. (2001) Proc Natl Acad Sci USA, 98, 2694-2697). Samples were quantitated on a BMG Fluorostar plate reader. A random subset of samples was checked on 2% agarose gel.

RT-PCR

Total RNA was extracted from PBMC before and after the stimulation with PHA, using Tri-Reagent (Sigma, Dorset, UK). First-strand cDNA synthesis was performed using random hexadeoxynucleotide primers and the first strand cDNA synthesis Kit (Amersham Biosciences, Amersham, UK). The CD45 cDNA was amplified using primers spanning the alternatively spliced CD45 exons—ex2 forward primer (5′-CGAAGCTTGCTGTTTCTTAGGGACACG-3′ (SEQ ID NO:11)) and ex7 reverse (5′-GTGAATTCCAGAAGGGCTCAGAGTGGT-3′ (SEQ ID NO:12)). The PCR conditions for amplification of CD45 cDNA included 4 min incubation at 94° C. followed by 30 reaction cycles (1 min at 94° C., 1 min at 55 C., 4 min at 72° C.) and final 16-min extension at 72° C. The PCR products were resolved on a Visigel Separation Matrix (Stratagene, La Jolla, Calif.). Bands were quantitated using Quality One Software (Bio-Rad, Hertfordshire, UK).

Flow Cytometric Analysis

PBMC were surface stained with the following mAbs against human CD45 isoforms—CD45R0-PE (clone UCHL1, Pharmingen, San Diego, Calif.), CD45R0-FITC (clone UCHL1, Pharmingen) CD45RB-FITC (clone PD7/26, Dako, Glostrup, Denmark), CD45RB-PE (clone MT4, Pharmingen), CD45RA-FITC (clone HI10, Pharmingen), CD45RA-PE (clone 4KB5, Dako) along with APC-conjugated CD3 (Pharmingen). For CD45RC (clone YTH80.103, BioSource, Camarillo, Calif.) analysis a second layer of affinity purified F(ab)′₂ goat anti-rat FITC or PE (Caltag, Silverstone, UK) was used. Isotype matched mAbs were used as controls. 10,000 events per sample were collected on a FACSCalibur flow cytometer (Becton Dickinson, Mountain View, Calif.) and analysed using WinMDI software. For stimulation studies PBMC were stimulated for 12 days with 1 μg/ml of PHA-P (Sigma).

Results

Identification of a Novel Point Mutation in Exon 6 of CD45 in Japanese and Korean Populations

To examine the CD45 locus for novel polymorphisms, we used DHPLC to detect mutations in the alternatively spliced exons 4(A), 5(B) and 6(C) of CD45, followed by sequencing of the target individuals.

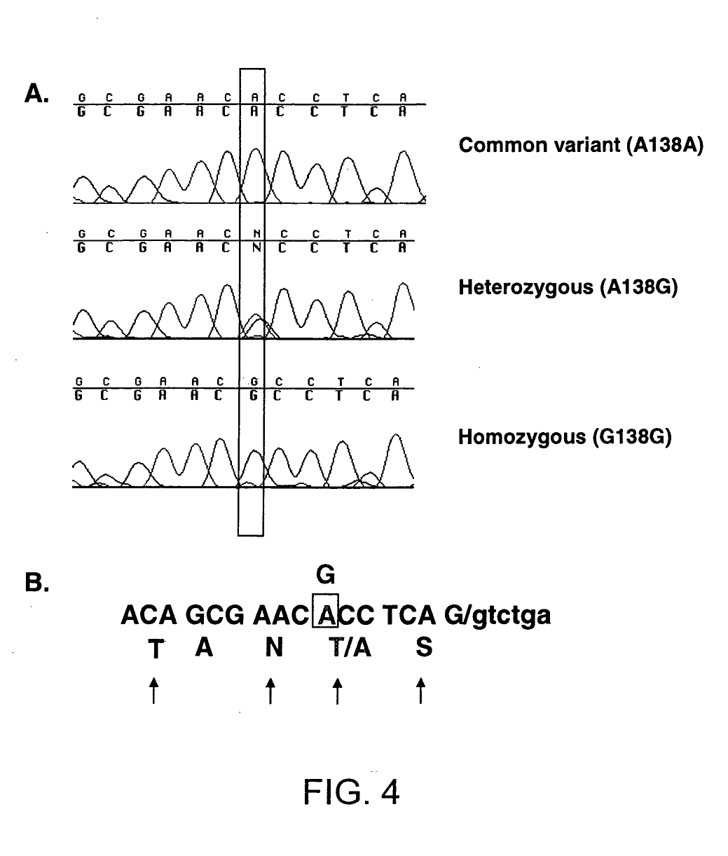

An A to G transversion at position 138 in exon 6 was found in Japanese samples. This is located 7 bp before the splice donor site at the 3′ end of exon 6, and results in a Threonine to Alanine semi-conservative amino acid change at position 47 of the CD45RC exon 6 (FIG. 4). Thr 47 is a potential O-linked glycosylation site (Van den Steen, P., Rudd, P. M., Dwek, R. A. & Opdenakker, G. (1998) Crit Rev Biochem Mol Biol 33, 151-208), but is also adjacent to an Asparagine and forms part of a consensus flanking sequence for an N-linked site as well. A substitution of this Thr may therefore lead to changes in the glycosylation of the extracellular domain of the molecule.

We used ARMS-PCR to detect the presence of the A138G variant (Table 2) and found 65 individuals out of 175 Japanese samples that carried the variant allele of which 9 were homozygotes for the G allele (allele frequency of 23.7%). The number of homozygotes was as expected according to the Hardy Weinberg Law. Note that the frequency of the A138G variant amongst the 49 Japanese patients with gynaecological cancer was within the normal range (17 heterozygotes and 2 homozygotes) and the presence of the variant did not correlate with any distinctive clinical manifestation. The high frequency of this allele in the Japanese population was further confirmed by re-sequencing all individuals indicated as carrying the allele. We also found 7 heterozygotes out of 48 Korean samples (allele frequency of 7.3%). No homozygotes have so far been found in this or other populations. The A138G variant was not detected in 209 Ugandan samples. We found 1 heterozygote out of 181 UK samples and 1 out of 72 Orkney samples. We also analysed samples from Asia and found 6 A138G heterozygotes in 65 Tatars (from Kazan and the Crimea) but none in 74 Russians from Tashkent. The 138G allele is also present in Chinese individuals (5 out of 12 A138G heterozygotes found among samples from Hong Kong Chinese and 3 out of 7 heterozygotes among samples from Beijing). 159 samples from Kagoshima in Southern Japan were also tested and revealed 114 A138A homozygotes, 36 A138G heterozygotes and 9 G138G homozygotes, a similar frequency of the 138G allele to that found previously in samples from Osaka.

We further compared the distribution of exon 6 A138G and exon 4 C77G variants, the latter being the only described common polymorphism in CD45 causing abnormal CD45 splicing (Table 2). The C77G variant was absent in samples from an African population (Ugandan) as has been previously shown (Tchilian, E. Z., et al., (2002) Immunogenetics 53, 980-983.) and was not detected amongst the Far Eastern Japanese and Korean populations. Interestingly the exon 4 C77G variant was found at a higher frequency (3.5%) in the United Kingdom Orkney islands than elsewhere, but no C77G homozygotes were found in the samples studied.

CD45 Isoform Expression on PBMC of Individuals with the Exon 6 A138G Variant

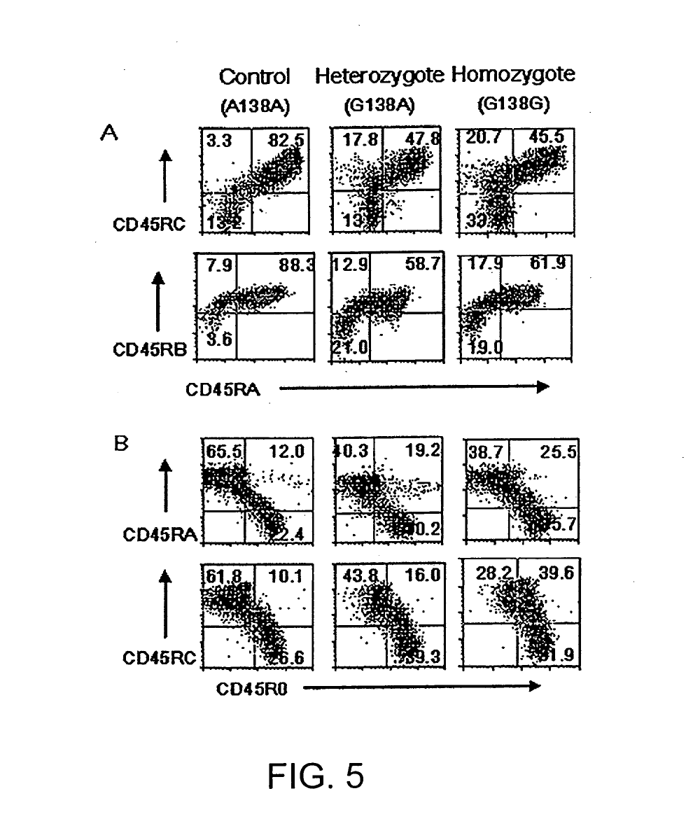

We next examined whether the novel A138G polymorphism affects CD45 isoform expression on the cell surface. Cryopreserved PBMC's from 4 healthy A138G heterozygotes, 4 G138G homozygotes and 4 common variant A138A homozygous controls were analysed by flow cytometry. CD45RA, CD45RB, CD45RC and CD45R0 antibodies were used to determine the expression of CD45 isoforms on these samples. There was a marked decrease in the proportion of cells expressing CD45RA and CD45RC or CD45RA and CD45RB isoforms in A138G positive individuals, with homozygotes showing a more extreme change (mean of 41.5% and 56.3% for CD45RA+CD45RC+ and CD45RA+CD45RB+ respectively) from the common variant controls 73.7% and 71.3% for CD45RA+CD45RC+ and CD45RA+CD45RB+) than the heterozygotes (49.6% and 55.9% for CD45RA+CD45RC+ and CD45RA+CD45RB+) (Table 3). There was a corresponding increase in CD45RA−CD45RC− or CD45RA−CD45RB− cells. Representative profiles are shown in FIG. 5A. A138G homozygotes had a higher proportion of cells expressing CD45R0 either in association with CD45RA (49.8% versus 22.8% in controls) and CD45RC (40.6% versus 11.2% in controls or in the absence of CD45RA (31.4% CD45R0+CD45RA− versus 20.5% in controls) and CD45RC (36.1% CD45R0+CD45RC− versus 20.1% in controls) (Table 3, FIG. 5B).

After 11 days stimulation with PHA, all of the CD3+ cells of the A138G homo- or heterozygous individuals showed very similar phenotypes to common variant control individuals with predominant expression of CD45R0 and CD45RB isoforms (data not shown).

No differences were observed in isoform expression on CD3 negative cells (not shown).

Taken together these data suggest that exon 6 A138G carriers have fewer T cells expressing isoforms containing the A, B or C exons (naive phenotype cells) and have more activated CD45R0+ cells compared to the common variant CD45 controls.

Effect on CD45 Splicing

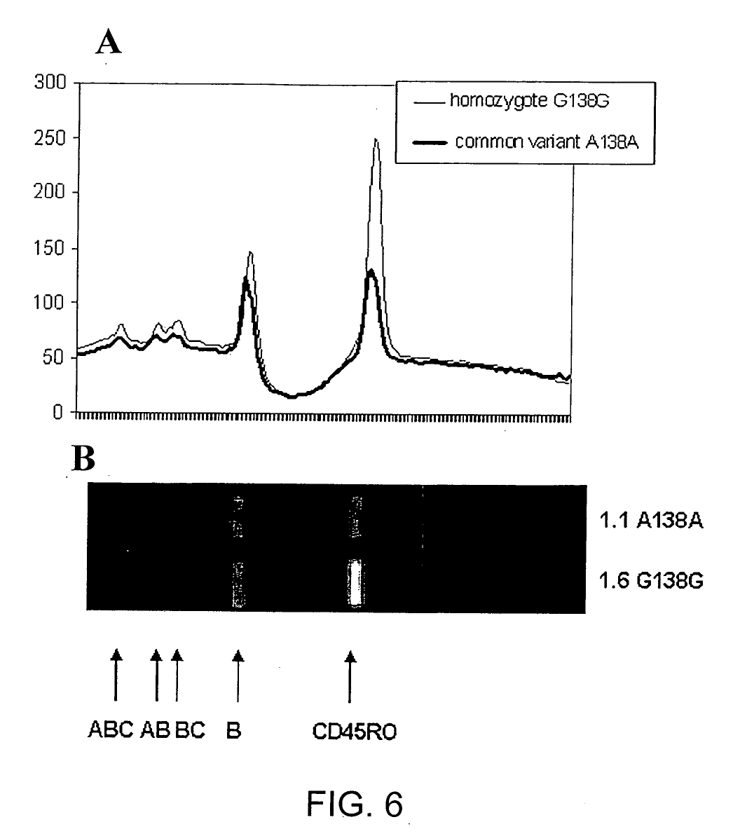

Because of the dramatic changes in the proportion of T cells expressing CD45 isoforms in A138G carriers, we next wanted to establish whether the exon 6 A138G variant interferes with CD45 splicing. RT-PCR analysis was performed on PBMC before and after stimulation with PHA. No qualitative differences in the expression of CD45 isoforms were observed between the homo-, and heterozygous A138G individuals and the controls at either time point (FIG. 6). However quantitation of the intensity of the bands showed a significant difference, in that the level of CD45R0 was increased in the mutated A138G gene when compared to the common variant.

These results suggest that the effect of this polymorphism is quantitative rather than qualitative with A138G carriers expressing more CD45R0 transcript compared to the controls.

Discussion

Described here is the identification of a polymorphism in exon 6 A138G of the gene encoding CD45 (PTPRC) which results in a semi-conservative amino acid substitution Thr47Ala in the extracellular domain of the CD45 molecule. This variant allele is present with a relatively high frequency in Korean (7.3%) and Japanese (23.7% populations, with homozygous individuals for the G allele amongst the Japanese. Although a thorough phenotypic and functional analysis has not yet been performed on A138G individuals, the results so far indicate that the carriers of the A138G mutation have a higher proportion of CD45R0+ T cells and a decrease in naive phenotype T cells expressing A, B and C isoforms.

The altered CD45 isoform expression is most likely caused by changes in alternative splicing, as shown by the increased levels of CD45R0 transcripts detected by RT-PCR in the A138G carriers. These findings are in agreement with earlier studies (Tsai, A. Y., Streuli, M. & Saito, H. (1989) Mol Cell Biol 9, 4550-4555) showing that mutations of nucleotides 134 to 144 at the most 3′ end of exon 6 resulted in mRNA that did not include exon 6 sequences. The exon 6 A138G mutation described here, exerts a more subtle quantitative effect and does not induce complete splicing out of exon C. It is plausible that in a similar way to the model proposed by Tsai et al., the A138G substitution may reduce the overall similarity of the splice site to the consensus sequence resulting in a less efficient recognition by the spliceosome. Alternatively, the exon 6 A138G change may induce alterations in exon splicing by disrupting regulatory elements within the exon itself (Smith, C. W. & Valcarcel, J. (2000) Trends Biochem Sci 25, 381-388). For example, the C77G polymorphism in exon 4 functions by disrupting an exon splicing silencer which normally represses the use of the 5′ splice site of exon 4 (Lynch, K. W. & Weiss, A. (2001) J Biol Chem 276, 24341-24347). Further studies using minigenes containing the mutation will be required to determine the precise mechanism for the altered CD45 expression in the A138G variant.

An alternative explanation for the observed phenotypic differences of the PBMC of A138G carriers might be that the variant results in the expression of a structurally altered CD45 molecule. Thus the A138G polymorphism results in the substitution of Thr47Ala, a potential glycosylation site for both O- and N-linked sugars and may therefore change the reactivity with carbohydrate dependent epitopes of anti-CD45 monoclonal antibodies (Pulido, R., Schlossman, S. F., Saito, H. & Streuli, M. (1994) J Exp Med 179, 1035-1040). Changes in the glycosylation would not only change the interactions with anti-CD45 antibodies, but may have important implications for the function of CD45 since the crucial contribution of carbohydrates to the regulation of CD45 isoform function has been documented. Several lectin like molecules have been shown to bind to carbohydrates of CD45, among other ligands. These include CD22 (Stamenkovic, I., Sgroi, D., Aruffo, A., Sy, M. S. & Anderson, T. (1991) Cell 66, 1133-1144), galectins (Perillo, N. L., Pace, K. E., Seilhamer, J. J. & Baum, L. G. (1995) Nature 378, 736-739; Walzel, H., Schulz, U., Neels, P. & Brock, J. (1999) Immunol Lett 67, 193-202; Symons, A., Cooper, D. N. & Barclay, A. N. (2000) Glycobiology 10, 559-563), mannose receptor (Martinez-Pomares, L., Crocker, P. R., Da Silva, R., Holmes, N., Colominas, C., Rudd, P., Dwek, R. & Gordon, S. (1999) J Biol Chem 274, 35211-35218) and serum mannan binding protein (Uemura, K., Yokota, Y., Kozutsumi, Y. & Kawasaki, T. (1996) J Biol Chem 271, 4581-4584; Baldwin, T. A. & Ostergaard, H. L. (2001) J Immunol 167, 3829-383). The CD45 ectodomain has also been suggested to influence CD45 engagement in cis interactions with TCR, CD4 and CD5 (Alexander, D. R. (1997) In Lymphocyte Signalling: Mechanism, subversion and manipulation, eds Harnett, M. M. & Rigley, K. P John Wiley & Sons Ltd., 107; Leitenberg, D., Y. Boutin, D. D. Lu, and K. Bottomly (1999) Immunity 10, 701; Dornan, S., et al., J Biol Chem 277, 1912-1918), but no direct binding between the CD45 ectodomain and another protein has been shown so far. Another proposed role for the CD45 extracellular domain is the regulation of dimersation and there is evidence that CD45 forms dimers on the cell surface (Majeti, R., et al., (2000) Cell 103, 1059-1070; Xu, Z. & Weiss, A. (2002) Nat Immunol 3, 764-771). These studies suggest that the structural differences caused by the A138G variant could affect the interactions of CD45 with potential ligands in cis and trans as well as dimerisation between CD45 isoform and might have functional consequences for the immune response.

It is interesting that the exon 4 C77G and exon 6 A138G variants have different distributions. This may suggest that variants arose independently after the emigration of ancestral humans from Africa. The high frequency of A138G variant in Japan suggests that it arose in the Far East and its low frequency elsewhere would confirm this. The maintenance of these CD45 variants in different human populations may be ascribed at present to selection or drift. Further functional and disease association studies may provide more convincing evidence for a selective effect, particularly of the novel A138G variant.

In Caucasoids the commonest polymorphism with an obvious phenotypic effect is the previously described C77G mutation in exon 4, which prevents normal splicing from high (CD45RA) to low (CD45RO) molecular weight isoform. We have previously shown that the frequency of the C77G variant allele in Northern Europe and North America is in the region of 0.85 to 1 6% and that it is absent in Africans (Tchilian, E. Z., et al., (2002) Immunogenetics 53, 980-983). The data presented here confirm the previously observed frequency in the UK (on a different set of UK samples) and the lack of this variant in African Ugandan populations (Tchilian, E. Z., et al., (2002) Immunogenetics 53, 980-983), and indicate a similar lack amongst the Far Eastern Japanese and Korean populations. The only exceptions are the Orcadians who have a higher allele frequency for the variant (3.5%). It would be interesting to analyse whether there is an association between the increased prevalence of exon 4 C77G variant and the high incidence of multiple sclerosis in the Orkney islands.

In summary the results suggest that individuals with the A138G variant have an increased proportion of T cells with an activated, memory or effector phenotype as determined by the increased proportion of CD45RO+ cells and reduced number of cells expressing the CD45 A, B and C isoform. The altered CD45 expression may therefore contribute to changes in interaction with potential ligands or homo- or heterodimerisation of the CD45 isoforms. Xu & Weiss (2002) Nat Immunol 3, 764-771 recently proposed a model suggesting that expression of the CD45R0 isoform in activated cells shifts the equilibrium of cell surface CD45 towards dimers, and acts as a negative regulator, contributing to the cessation of the immune response. Increased expression of the CD45R0 isoform caused by A138G polymoiphism would promote this negative regulation, resulting in a less vigorous immune response which may reduce the risk of autoimmune disease in A138G carriers. Alternatively, the high proportion of CD45R0+ cells may indicate that these individuals have a larger memory population and can make vigorous recall responses to pathogens. The high frequency of this allele in Japan and Korea may indicate that it confers a survival advantage.

TABLE 2
Frequency of CD45 exon 4 C77G and exon 6
A138G alleles in different populations. Homozygotes
are shown in brackets.

| | | Exon 6 (A138G) | | Exon 4 (C77G) | |
|---|---|---|---|---|---|
| | Total | | Allele | | Allele |
| population | Number | A138G | Frequency % | C77G | Frequency % |
| Japanese | 175 | 65(9) | 23.7 | 0 | 0 |
| Korean | 48 | 7 | 7.3 | 0 | 0 |
| United | 181 | 1 | 0.3 | 4 | 1.1 |
| Kingdom | | | | | |
| Orkney | 72 | 1 | 0.7 | 5 | 3.5 |
| Tatar | 65 | 6 | 4.6 | 3 | 2.3 |
| Russian/ | 74 | 0 | 0 | 3 | 2 |
| Tashkent | | | | | |
| Ugandan | 209 | 0 | 0 | 0 | 0 |

TABLE 3
CD45 isoform expression on CD3+ cells from
A138G and control individuals. Means and standard
deviations of data expressed as the percentage of CD3+
T cells from 4 homozygous (G138G), 4 heterozygous
(A138G) and 4 homozygous for the common variant
(A138A) control individuals.

| | Control | Heterozygote | Homozygote |
|---|---|---|---|
| Cell Subset | (A138A) | (G138A) | (G138G) |
| CD45RA+CD45RC+ | 73.7 +/− 12.0 | 49.6 +/− | 41.5 +/− 5.5 |
| CD45RA−CD45RC− | 18.7 +/− 8.4 | 34.5 +/− | 36.0 +/− 3.7 |
| CD45RA+CD45RB+ | 71.3 +/− 14.1 | 55.9 +/− | 56.3 +/− 5.5 |
| CD45RA−CD45RB− | 10.1 +/− 7.8 | 20.7 +/− | 21.8 +/− 2.1 |
| CD45RC+CD45RO+ | 11.2 +/− 5.4 | 13.4 +/− | 40.6 +/− 5.7 |
| CD45RC+CD45RO+ | 20.1 +/− 6.6 | 32.0 +/− | 36.1 +/− 5.0 |
| CD45RA+CD45R0+ | 29.8 +/− 22.8 | 56.5 +/− 12.5 | 49.8 +/− 23.3 |
| CD45RA+CD45R0+ | 20.5 +/− 78 | 31.8 +/− 6.0 | 31.4 +/− 3.6 |

### Example 5—Associations Between A138G and Disease

Methods

Materials

DNA samples from 175 Graves' and 126 Hashimoto's patients were obtained through Osaka City University Hospital. Hyperthyroidism due to Graves' disease was diagnosed on the basis of history and signs of hyperthyroidism with diffuse goiter and the laboratory findings, including elevated serum free T4 and free T3 concentrations, undetectable serum thyroid stimulating hormone (TSH), and positive TSH receptor antibody. Hashimoto's thyroiditis was diagnosed by positive thyroglobulin and/or thyroid peroxidase antibodies, reduced echogenicity on thyroid ultrasound, and normal or elevated TSH level. 94 Hepatitis B and 124 Hepatitis C samples were collected in the outpatient clinic of Osaka City University Hospital. All of the 94 Hepatitis B patients were infected at birth by transmission from their mothers and were positive for Hepatitis B surface antigen. The Hepatitis C patients were infected later in life and were all positive for antibodies to HCV antigen. In all samples HCV RNA was detected, except for four patients who have cleared the virus (two of these were A138G heterozygous and one G138G homozygous). As control samples 175 Japanese genomic DNA's collected from Osaka City University Medical School were used as previously described (Stanton, T. et al. Proc Natl Acad Sci USA 100, 5997-6002. (2003)). Approval was obtained by the Ethical committee of the City University Graduate School of Medicine Osaka and the patients gave consent for the study.

Amplification Refractory Mutation System (ARMS) PCR

To detect carriers of the exon 6 A138G mutations, we used amplification refractory mutation system (ARMS)PCR, with two separate reaction mixes, containing one forward primer and one of the two reverse primers as previously described (Example 4 and Stanton, T. et al. Proc Natl Acad Sci USA 100, 5997-6002. (2003)). The presence of the 138G variant allele in all of the samples was confirmed by sequencing.

Flow Cytometric Analysis

Phenotypic analysis was performed on PBMC from 6 healthy A138A controls and 4 healthy G138G homozygous carriers. Cells from 4 A138G heterozygotes were also analysed (data not shown). The ages of all subjects in this study were between 27 and 58 years. 2×10⁵ PBMC were stained with either allophycocyanine (APC)-conjugated CD4 (S3.3, Caltag, Silverstone, UK) or CD8-APC (clone RPA/T8, Pharmingen, San Diego, Calif.) along with fluorescein isothyocyanate (FITC)-conjugated CD45RA (clone HI10, Pharmingen) and phycoerythrin (PE)-conjugated CD45R0 (clone UCHL1, Pharmingen) mAbs in a single step at 4° C. for 20 minutes and washed with PBS, containing 0.2% BSA. The following reagents and antibodies were also used to stain cell suspensions: CD11a-FITC (G43-25B), CD28-FITC (CD28.2), CD62L-FITC (Dreg56), CD95-FITC (DX2), CCR7 (2H4) were all from Pharmingen, CD27-FITC (LT27) (Serotec, Kidlington, UK).

For intracytoplasmic staining 1×10⁵ PBMC per well were incubated in U-bottom 96-well plates in 200 μl of RPMI1640+10% FCS in the presence of 50 ng/ml phorbol myristate (PMA) and 0.5 μg/ml ionomycin. GolgiPlug (Pharmingen) was added after 2 hours and cells incubated for an additional 12 hours. The cells were surface labelled with CD4-APC or CD8-APC antibodies as described above and permeabilised with 40 μl Permafix (OrthoDiagnostic, UK) for 40 min in the dark. The cells were washed and stained with FITC conjugated IFN-gamma antibody (clone 25723.11, Pharmingen) for 30 min at room temperature.

Isotype matched mAbs were used as controls. 10,000 or 50,000 events per sample were collected on a FACSCalibur (Becton Dickinson, Mountain View, Calif.) and analysed using WinMDI software.

Statistical Analysis

Chi-Square test, using Yates continuity correction to allow for small numbers was used to analyse the disease association of the 138G variant allele. For comparison of phenotypic analyses between cell subsets in A138G and control individuals, Student's t-test, assuming equal variance, was used.

Results

We studied the frequency of the 138G variant in cohorts of Japanese patients with thyroid autoimmune conditions. In Hashimoto's thyroiditis (HT) cellular and humoral responses to thyroid antigens lead to destruction of the organ and hypothyroidism while Graves' disease (GD) is characterised by hyperthyroidism, caused by stimulatory thyrotropin receptor antibodies. We analysed 126 Hashimoto patients and found 50 A138G heterozygotes (allele frequency, 19.8%), comparable to the frequency in the control population (23.7%) (Table 4). Interestingly no G138G homozygotes were detected amongst the Hashimoto samples although 5 were expected according to the Hardy-Weinberg Law (p=0.02) a result suggesting a recessive effect of the 138G allele in HT. We found 31 heterozygotes (frequency 9%) and no homozygotes out of 175 Graves' samples. The difference between the controls and GD is very significant (p<0.01). In contrast to HT, this suggests a dominant effect for the 138G allele in GD.

We further analysed the frequency of the variant in two important viral infections—Hepatitis B (HBV) and Hepatitis C. We found 20 A138G heterozygotes and no homozygotes among 94 Hepatitis B carrier samples (allele frequency, 10.6%). The difference between the controls and HBV is significant (p=0.01). The reduction in heterozygotes suggests a dominant effect of the 138G allele in this disease. In HCV, we found 34 A138G heterozygotes and 7 G138G homozygotes in 124 samples, figures that are as expected according to Hardy-Weinberg law. Taken together these results show a significant dominant protective effect of the 138G allele in GD and HBV infection, but a recessive protective effect in HT.

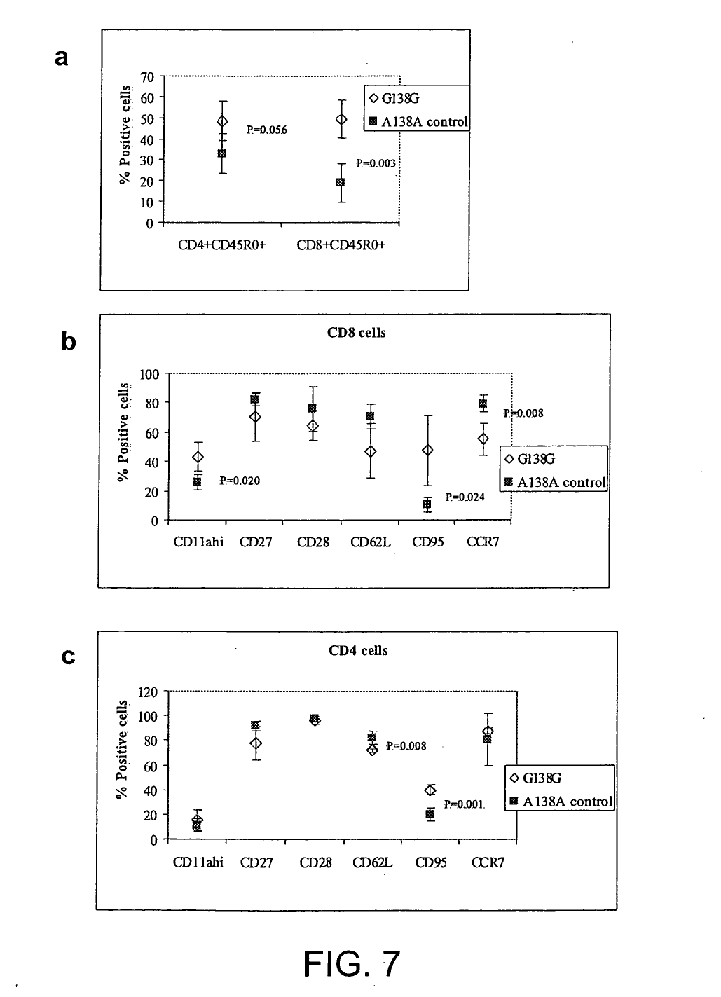

We next sought evidence for altered immune phenotype and function in individuals carrying the 138G allele. We examined whether the altered pattern of CD45 isoforms in 138G individuals affects the expression of other leucocyte antigens associated with differentiation of T cells. Peripheral blood mononuclear cells (PBMC's) from healthy G138G. homozygotes, A138G heterozygotes and A138A control homozygotes were analysed by flow cytometry. All the G138G variant samples showed the previously described increased proportion of CD45R0+ T cells (Stanton, T. et al. Proc Natl Acad Sci USA 100, 5997-6002. (2003)); among CD8 cells mean 49.4+/−8.9%, compared to 18.9+/−9.3% in controls (p=0.003) (FIG. 7a), and in the CD4 subset mean 48.4+/−9.3% versus 32.8+/−9.3% in A138A controls (p=0.056). A138G heterozygotes show an intermediate CD45R0+ phenotype for CD8 and CD4 cells (data not shown). Furthermore the G138G individuals exhibit decreased expression of CD27, CD28, CD62L and CCR7 and increased expression of CD11a and CD95 (FIG. 7b). Less exaggerated changes in expression of these markers were detected in the CD4 (FIG. 7c). These changes suggest that the most prominent effect in 138G individuals is an increase in the proportion of activated/memory T cells.

We next analysed cytokine production in PBMC from 138G individuals. Intracytoplasmic flow cytometric analysis showed that stimulated G138G cells have a significantly higher frequency of CD4 and CD8 cells able to secrete interferon-gamma (IFN-gamma) (Table 5). Heterozygotes showed an intermediate frequency of cytokine-producing cells in both T cell subsets. These results show that expression of the variant 138G allele is associated with increased production of the Th1 cytokine IFN-gamma by CD4 and CD8 cells.

TABLE 4
Frequency of CD45 exon 6 A138G alleles in
control and disease groups.

| | | | A138G | G138G |
|---|---|---|---|---|
| Disease | Total | | (allele | (allele |
| Group | number | A138A | frequency %) | frequency %) |
| Control | 176 | 111 | 56 (23.7%) | 9 |
| Hashimoto | 126 | 76 | 50 (19.8%) | 0* |
| Graves' | 175 | 144 | 31 (9%)** | 0 |
| Hepatitis B | 94 | 74 | 20 (10.6%)*** | 0 |
| Hepatitis C | 124 | 83 | 34 (19.4%) | 7 |

Statistically significant differences with controls with *p = 0.02, **p < 0.01 and ***p = 0.0124.

TABLE 5
Frequency of IFN-gamma producing CD4 and CD8
T cells of individuals with different 138G alleles

| | % CD4 cells | % CD8 cells |
|---|---|---|
| | expressing IFNγ | expressing IFNγ |
| G138G | 30.7 +/− 4.1* | 35.3 +/− 11.6⁺ |
| A138G | 28.6 +/− 4.3** | 17.8 +/− 4.6⁺⁺ |
| A138A | 18.9 +/− 1.8 | 9.7 +/− 2.8 |

Four individuals of each genotype were studied.
Differences between G138G or A138G individuals and A138A controls were analysed by Student's T test.
*p = 0.002,
**p = 0.006,
⁺p = 0.005,
⁺⁺p = 0.024.

Discussion

There are several possible explanations for the effect of the 138G variant. An important factor in the pathogenesis of autoimriune diseases is a change in the balance between Th1 cytokines which promote cell mediated immunity and Th2 cytokines, which promote humoral immunity. In GD there is a shift toward Th2 cytokine responses (Kocjan, T., et al., Pflugers Arch 440, R94-95. (2000); Ludgate, M. & Baker, G. Clin Exp Immunol 127, 193-198. (2002)), while Hashimoto's patients show Th1 activation (Mazziotti, G. et al. Eur J Endocrinol 148, 383-388. (2003)). It is likely that the increased IFN-gamma production in 138G carriers would counteract the Th2 cytokine deviation in GD. Furthermore is has been suggested that activated (IFN-gamma-producing) CD8 cells may reduce the pathogenic Th2 dominance in GD (Bartlett, S. et al., (2001) Proc Natl Acad Sci USA 98, 2694-2697). In contrast the increased IFN-gamma production in the 138G variant might not affect the disease course and already polarised Th1 balance in HT. However, the lack of G138G homozygotes in HT suggests the possibility of a specific effect in homozygotes which needs further investigation.

The contribution of CD8 cells to the control of HBV infection is well documented (Thimme, R. et al. J Virol, Vol. 77, 68-76. (2003)). In addition to clearance of infected cells by cytolytic CD8 cells, the anti-viral effect of IFN-gamma produced by these cells has been shown to be an important protective mechanism (Guidotti, L. G. & Chisari, F. V. Annu Rev Immunol, 19, 65-91. (2001)). It is very likely that the increased proportions of activated T cells and IFN-gamma production in 138G neonates would limit amplification of the virus. Furthermore it has been suggested that neonates have Th2 biased immune responses (Chen, N. & Field, E. H. Transplantation, 59, 933-941. (1995); Barrios, C. et al. Eur J Immunol, 26, 1489-1496. (1996)) and is possible that the prevalence of Th1 cytokines in 138G infants would be beneficial at this early stage of life in controlling the HBV infection, while it would not have such a significant impact later in life. This might be the case for the Hepatitis C cohort we have studied. Whatever the mechanism, comparisons of immune responses of individuals carrying or lacking the 138G allele may provide insights into the molecular mechanisms underlying the interactions between HCV and HBV and IFN-gamma.

Although there have been previous reports of altered CD45 isoform expression in disease (Sempe, P. et al. Int Immunol, 5, 479-489 (1993); Renno, T. et al. Int Immunol, 6, 347-354 (1994); Subra, J. F. et al. J Immunol, 166, 2944-2952 (2001)), we now provide evidence that genetic variants affecting CD45 isoform expression are associated with autommunity and viral infection, suggesting a crucial role of CD45 in modulating immune responses. It is conceivable that the original selection for the 138G CD45 variant may have been with respect to pathogen resistance and what we see now is a residue of this after the pathogen effect has gone. The high frequency of 138G individuals (˜40% in Japan) suggests that the allele is likely to affect susceptibility and pathogenesis in other autoimmune and infectious diseases.

### Example 6—Identification of Novel A54G Polymorphism

Materials and Methods

Materials

269 Ugandan DNA samples from the Entebbe cohort (160 HIV-seropositive and 109 seronegative) were provided by Pontiano Kaleebu, Christine Watera, Jimmy Whitworth and Adrian Hill. 181 UK, 175 Japanese and 48 Korean genomic DNA samples were obtained as previously described (Stanton, T., et al., 2003, Proc Natl Acad Sci USA 100:5997.). 40 Malawian samples were provided by Paul Fine and Hazel Dockrell. All of these samples were HIV negative. Ethical approval was obtained. PBMC samples for FACS analysis were obtained from one A54G and two A54A control Ugandans only. All these were HIV positive.

Denaturing High-Performance Liquid Chromatography (DHPLC) and Sequencing

Genomic DNA was amplified by PCR by using the following primers flanking exon 4: ex4 forward (5′-CATATTTATTTTGTCCTTCTCCCA-3′ (SEQ ID NO:6), ex4 reverse (5′-GTGCAGAAATGCAGGAAAT-3′ (SEQ ID NO:9)) generating a product of 384 bps. A two-stage, 34-cycle PCR was performed, with an initial 10-min denaturation at 95° C., then 14 cycles of 30 s at 95° C., 30 s at 61.5° C. minus 0.5° C. per cycle, and 30 s at 72° C., followed by 20 cycles of 30 s at 95° C., 30 s of annealing at 54° C., 30 s at 72° C., and a final 7-min extension at 72° C. PCRs were performed in a volume of 50 μl, containing 10 pmol of each primer, 200 μM dNTP, 2.5 mM MgCl₂, and 0.5 units of AmpliTaq Gold (Perkin-Elmer) in 1×KCl Perkin-Elmer buffer II. 5 μl of the PCR product was resolved on 2% agarose to test product size, and the remaining product was denatured for 4 min at 95° C., followed by 42 cycles of 1 min at 95° C., dropping by 1.6° C. per cycle to 28.8° C. to hybridize. Products were run on the DHPLC machine (WAVE, Transgenomic, Crewe, U.K.). Purified PCR products were subjected to automated sequencing by using the same primers as for DHPLC.

Flow Cytometric Analysis

PBMC were surface stained with the following mAbs against human CD45 isoforms—CD45R0-PE (clone UCHL1, Pharmingen, San Diego, Calif.), and CD45RA-FITC (clone HI10, Pharmingen), along with APC-conjugated CD3 (Pharmingen). 10,000 events per sample were collected on a FACSCalibur flow cytometer (Becton Dickinson, Mountain View, Calif.) and analysed using WinMDI software.

Results

Using denaturing high performance liquid chromatography, an A to G transversion was found at position 54 in exon 4. This A54G variant results in a Thr to Ala semiconservative amino acid substitution at position 19 of the CD45RA exon 4.

The A54G variant was found in Ugandan samples, but was absent amongst Far Eastern (175 Japanese and 48 Koreans), UK Caucasoids (181 UK and 72 Orkneys) and African (40 Malawian) populations (Table 6).

We investigated the distribution of the new 54G allele in a Ugandan (Entebbe) cohort of HIV seropositive and seronegative individuals. We found six A54G heterozygotes out of 160 HIV seropositive (allele frequency, 1.87%) and one heterozygote out of 109 HIV seronegative controls individuals (0.45%). The presence of the variant allele was confirmed by sequencing in all samples. The difference between the controls and HIV infected individuals is very similar to that observed by us for the C77G variant in UK HIV infected individuals, with about four fold higher frequency in patients ver-sus controls (p=0.24 by Fisher exact test).

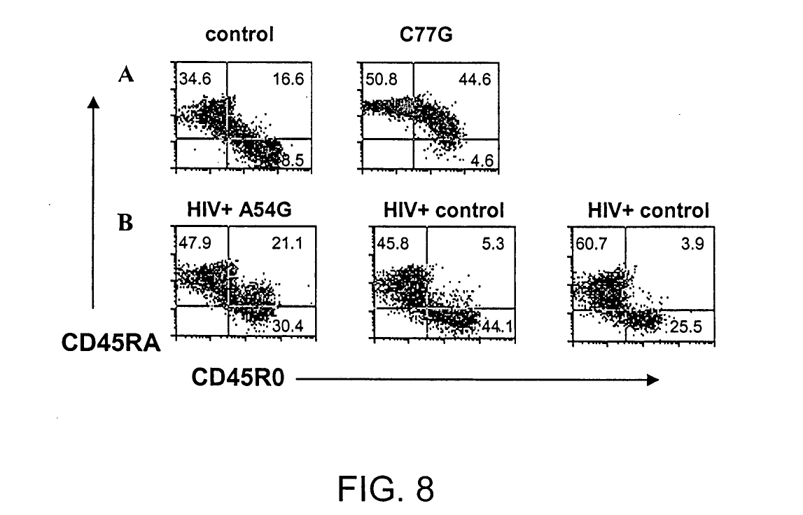

Since exon 4 C77G and C59Avariants have been shown to alter CD45 splicing, we next examined whether the A54G polymorphism affects CD45 isoform expression on the cell surface. Cryopreserved PBMC from one A54G and 2 A54A control individuals were analysed, all three were HIV seropositive. PBMC samples were gated on CD3 T cells and CD45RA and CD45R0 expression analysed. The normal pattern of CD45 expression is characterised by the presence of CD45RA+ cells lacking CD45R0 expression and CD45R0+ cells lacking CD45RA (FIG. 8A), while the abnormal C77G pattern is characterised by the absence of the CD45RA− population and an increased proportion of double positive RA/RO cells (44.6 versus 16.%). The A54G individual showed an increase proportion of double positive RA+R0+ cells, compared to the two Ugandan HIV A54A controls (21.1% versus 5.3% and 3.9%) (FIG. 8B), although there is no clear deficit of CD45RA− cells as in C77G.

TABLE 6
Frequency of A54G variant in Ugandan HIV
seropositive and seronegative individuals.

| Polymorphism | Population | Allele frequency (%) |
|---|---|---|
| Exon 4 A54G | HIV Ugandans | 1.87 |
| | Control Ugandans | 0.45 |
| | UK & Orkney | 0 |
| | Japanese | 0 |
| | Koreans | 0 |
| | Malawi | 0 |

### Example 7—Phenotypic Analysis of C77G Polymorphism

The following study examined whether the abnormal pattern of CD45 isoform expression affects other aspects of leukocyte phenotype.

Materials and Methods

Flow Cytometric Analysis

PBMC were stimulated for 12 days with 1 μg/ml of PHA-P (Sigma, St Louis, Mo.) adding IL-2 on day 10. Flow cytometric analysis of CD45 variant splicing was performed as previously described (Tchilian E Z, et al., ibid.). Briefly, 2×10⁵ PBMC were stained with either APC-conjugated CD4 (S3.3, Caltag, Silverstone, UK) or CD8-APC (clone RPA/T8, Pharmingen, San Diego, Calif.) along with FITC-conjugated CD45RA (clone HI10, Pharmingen) and PE-conjugated CD45R0 (clone UCHL1, Pharmingen) mAbs in a single step at 4° C. for 20 minutes and washed with PBS, containing 0.2% BSA. The following reagents and antibodies were also used to stain cell suspensions: CD11a-FITC (G43-25B), CD28-FITC (CD28.2), CD44-FITC (G44-26), CD62L-FITC (Dreg56), CD95-FITC (DX2), CCR7 (2H4) were all from PharMingen, CD62L-FITC (LAM1-116) (Ancell, Bayport, USA), CD69-FITC (CH14) (Caltag, Silverstone, UK), HLA-DR-FITC (TU36) (Caltag), CD25-FITC (ACT-1) (Dako), CD4-FITC (Dako), CD27-FITC (LT27) (Serotec, Kidlington, UK). Isotype matched mAbs were used. as controls. 10,000 or 50,000 events per sample were collected on a FACSCalibur (Becton Dickinson, Mountain View, Calif.) and analysed using WinMDI software.

Results

Cryopreserved PBMC's from healthy individuals known to carry the 77G mutation and cryopreserved normal control cells were analysed by flow cytometry. No apparent differences in the proportion of CD3, CD4, CD8, CD14 and CD19 cells were observed between the individuals with the 77G variant and wild type CD45 (data not shown). All of the 77G samples showed the previously described typical pattern of CD45 isoform expression on both CD4 and CD8 cells. Even after 12 days stimulation with PHA and IL-2, neither CD4 nor CD8 cells of 77G individuals were able to switch to expression of only the CD45R0 isoform (data not shown). However, it is noteworthy that the CD8 cells of individuals with the 77G mutation have more CD45RA single positive cells (mean 75%) compared to normal individuals (mean 58%) (p=0.001 for 6 77G carriers and 6 controls). In contrast the proportions of CD45RA versus CD45R0 or CD45RA/R0 double positive cells are similar among CD4 cells from 77G and control samples.

Because of the strikingly altered proportions of CD45RA positive versus CD45R0 or CD45RA/R0 double positive cells among CD8 cells from 77G and control individuals we next examined the expression of various cell surface markers associated with lymphocyte activation, analysing them in the CD45R0+ and CD45R0− subsets.

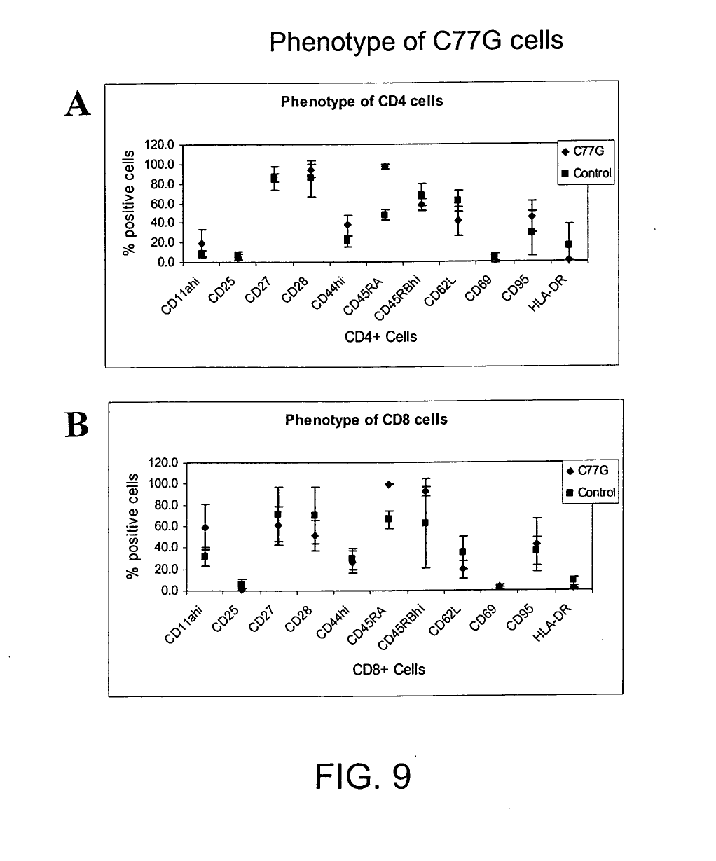

FIG. 9 illustrates FACs analysis of PBMC from 4 77G and 4 control normal individuals. Staining for a panel of markers has been analysed after gating on CD4 and CD8 T lymphocytes. In CD8 T cells no statistically significant differences were observed in the expression of the adhesion molecule CD44, the costimulatory molecule CD28, cytokine and chemokine receptors CD25 and CCR7 and the activation markers CD69 and HLA-DR. However, an increased frequency of CD8 cells expressing high levels of the adhesion molecule CD11a (CD11ahi) was detected in the (enlarged) CD45R0− subset (p=0.025). In the CD45R0+ subset the expression of CD27, CD62L and CD95 was significantly decreased in C77G individuals compared to controls. The differences in CD62L expression were confirmed with a different CD62L antibody (clone LAM1-116, data not shown), suggesting that the observed variances were not due to differential glycosylation of surface molecules in C77G and control cells.

Summary

Both CD4 and CD8 T cells show a decreased percentage of CD62L stained cells and an increased percentage of CDllahi and CD95 positive cells in 77G individuals as compared to controls. These changes indicate that there are increased numbers of activated lymphocytes amongst both populations but the effects are more obvious in the CD8 population.

Equivalents

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

We claim:

1. A method of screening a human subject for susceptibility to viral infection and/or pre-disposition to developing severe disease following viral infection, which method comprises screening for the presence or absence in the genome of the subject of one or more polymorphic variants or mutations in the gene encoding CD45 or of one or more polymorphic variants in linkage disequilibrium with or in close physical proximity to a polymorphic locus in the gene encoding CD45.

2. A method according to claim 1 wherein the mutation in the gene encoding CD45 is characterised in that subjects carrying at least one mutant allele exhibit altered CD45 splicing resulting in a reduction in the proportion of the T cell population carrying the CD45RO splice variant but lacking CD45RA expression as compared to subjects not carrying a mutant allele, wherein subjects having at least one mutant allele are scored as being more susceptible to viral infection and/or more pre-disposed to developing severe disease following viral infection, as compared to subjects who do not carry a mutant allele.

3. A method according to claim 2 wherein the mutation in the gene encoding CD45 is the C77G mutation, wherein subjects having at least one 77G mutant allele are scored as being more susceptible to viral infection and/or more pre-disposed to developing severe disease following viral infection, as compared to subjects who do not carry a mutant allele.

4. A method according to claim 2 wherein the mutation in the gene encoding CD45 is the C59A mutation, wherein subjects having at least one 59A mutant allele are scored as being more susceptible to viral infection and/or more pre-disposed to developing severe disease following viral infection, as compared to subjects who do not carry a mutant allele.

5. A method according to claim 2 wherein the mutation in the gene encoding CD45 is the A54G mutation, wherein subjects having at least one 54G mutant allele are scored as being more susceptible to viral infection and/or more pre-disposed to developing severe disease following viral infection, as compared to subjects who do not carry a mutant allele.

6. A method according to claim 1 which comprises screening for the presence or absence in the human subject of a polymorphic variant or mutation in linkage or linkage disequilibrium with at least one mutation in the gene encoding CD45 selected from the group consisting of the C77G mutation, the C59A mutation and the A54G mutation, wherein subjects having at least one allele in linkage or linkage disequilibrium with the 77G mutant allele and/or the 59A mutant allele and/or the 54G mutant allele are scored as being more susceptible to viral infection and/or more pre-disposed to developing severe disease following viral infection, as compared to subjects who do not carry an allele in linkage or linkage disequilibrium with the 77G mutant allele and/or the 59A mutant allele and/or the 54G mutant allele.

7. A method according to claim 2 wherein the viral infection is infection with a virus selected from the group consisting of: human immunodeficiency viruses, HIV-1, Epstein-Barr virus and poliovirus.

8. A method according to claim 1 wherein the mutation in the gene encoding CD45 is characterised in that subjects carrying at least one mutant allele exhibit altered CD45 splicing resulting in an increase in the proportion of the T cell population carrying the CD45RO splice variant but lacking CD45RA expression as compared to subjects not carrying a mutant allele, wherein subjects having at least one mutant allele are scored as being less susceptible to viral infection and/or pre-disposed to developing less severe disease symptoms following viral infection, as compared to subjects who do not carry a mutant allele.

9. A method according to claim 8 wherein the mutation in the gene encoding CD45 is the A138G mutation, wherein subjects having at least one 138G mutant allele are scored as being less susceptible to viral infection and/or pre-disposed to developing less severe disease symptoms following viral infection, as compared to subjects who do not carry a 138G mutant allele.

10. A method according to claim 1 which comprises screening for the presence or absence in the human subject of a polymorphic variant or mutation in linkage or linkage disequilibrium with the A138G mutation in the gene encoding CD45, wherein subjects having at least one 138G mutant allele are scored as being less susceptible to viral infection and/or pre-disposed to developing less severe disease symptoms following viral infection, as compared to subjects who do not carry an allele in linkage or linkage disequilibrium with the 138G mutant allele.

11. A method according to claim 8 wherein the viral infection is infection with hepatitis B virus.

12. A method of screening a human subject for an altered immune response capability, which method comprises screening for the presence or absence in said subject of a mutation in the gene encoding CD45, which mutation is characterised in that subjects carrying at least one mutant allele exhibit altered CD45 splicing resulting in an increase in the proportion of the T cell population carrying the CD45RO splice variant but lacking CD45RA expression as compared to subjects not carrying a mutant allele, wherein subjects having at least one mutant allele are scored as having altered immune response capability.

13. A method according to claim 12 wherein the mutation in the gene encoding CD45 is the A138G mutation, wherein subjects having at least one 138G mutant allele are scored as having altered immunological response capability, as compared to subjects who do not carry a 138G mutant allele.

14. A method according to claim 12 wherein the altered immune response capability is a more vigorous response to infection by pathogenic substances or organisms, wherein subjects having at least one mutant allele are scored as exhibiting a more vigorous response to pathogenic substances or organisms than subjects not having a mutant allele.

15. A method according to claim 12 wherein the altered immune response capability is increased production of interferon-gamma by CD4 and/or CD8 T cells, wherein subjects having at least one mutant allele are scored as exhibiting increased production of interferon-gamma by CD4 and/or CD8 T cells as compared to subjects not having a mutant allele.

16. A method according to claim 12 wherein the altered immune response capability is an increase in the proportion of T cells having the activated, memory or effector phenotype, wherein subjects having at least one mutant allele are scored as exhibiting an increased proportion of T cells having the activated, memory or effector phenotype as compared to subjects not having a mutant allele.

17. The method of claim 12 for use in evaluating susceptibility of a human subject to autoimmune disease, wherein subjects having at least one mutant allele are scored as having altered immune response capability and therefore having reduced susceptibility to autoimmune disease, as compared to subjects not having a mutant allele.

18. The method of claim 12 for use in evaluating the likely severity of autoimmune disease symptoms in a human subject, wherein subjects having at least one mutant allele are scored as having altered immune response capability and therefore likely to exhibit less severe autoimmune disease symptoms, as compared to subjects not having a mutant allele.

19. The method according to claim 17 or claim 18 wherein the autoimmune disease is Graves' disease or Hashimoto's thyroiditis.

20. The method of claim 12 for use in evaluating susceptibility of a human subject to viral infection, wherein subjects having at least one mutant allele are scored as having altered immune response capability and therefore having reduced susceptibility to viral infection, as compared to subjects not having a mutant allele.

21. The method of claim 12 for use in evaluating the likely severity of disease symptoms following viral infection in a human subject, wherein subjects having at least one mutant allele are scored as having altered immune response capability and therefore likely to exhibit less severe disease symptoms following viral infection, as compared to subjects not having a mutant allele.

22. The method according to claim 20 or claim 21 wherein the viral infection is infection with hepatitis B virus.

23. The method of claim 12 for use in evaluating susceptibility of a human subject to allergy or atopic disease, wherein subjects having at least one mutant allele are scored as having altered immune response capability and therefore less susceptible to allergy or atopic disease as compared to subjects not having a mutant allele.

24. The method of claim 12 for use in predicting the likely response of a human subject to a vaccine.

25. A method according to claim 24 wherein the vaccine is an anti-tumour vaccine.

26. A method of screening a human subject for susceptibility to viral infection and/or pre-disposition to developing severe disease following viral infection which comprises evaluating the pattern of CD45 mRNA expression in the subject, wherein the presence of an abnormal pattern of CD45 mRNA expression characterised by reduced splicing out of exon 4 of the CD45 mRNA and a quantitative decrease in amount of CD45RO transcript is taken as an indication that the subject is susceptible to viral infection and/or pre-disposed to developing severe disease following viral infection.

27. A method according to claim 26 wherein the abnormal pattern of CD45 mRNA expression is that associated with the presence of a 77G mutant allele of the gene encoding CD45, wherein detection of the abnormal pattern of CD45 mRNA expression is taken as an indication that the subject is more susceptible to viral infection and/or more pre-disposed to developing severe disease following viral infection, as compared to subjects who do not carry a 77G mutant allele.

28. A method according to claim 26 wherein the abnormal pattern of CD45 mRNA expression is that associated with the presence of a 59A mutant allele of the gene encoding CD45, wherein detection of the abnormal pattern of CD45 mRNA expression is taken as an indication that the subject is more susceptible to viral infection and/or more pre-disposed to developing severe disease following viral infection, as compared to subjects who do not carry a 59A mutant allele.

29. A method of screening a human subject for susceptibility to viral infection and/or pre-disposition to developing severe disease following viral infection which comprises evaluating the pattern of CD45 mRNA expression in the subject, wherein the presence of an abnormal pattern of CD45 mRNA expression characterised by a quantitative increase in the level of expression of the CD45RO transcript is taken as an indication that the subject is not susceptible to viral infection and/or is pre-disposed to developing less severe disease following viral infection.

30. A method according to claim 29 wherein the abnormal pattern of CD45 mRNA expression is that associated with the presence of a 138G mutant allele of the gene encoding CD45, wherein detection of the abnormal pattern of CD45 mRNA expression is taken as an indication that the subject is less susceptible to viral infection and/or pre-disposed to developing less severe disease following viral infection, as compared to subjects who do not carry a 138G mutant allele.

31. A method of screening a human subject for an altered immune response capability, which method comprises evaluating the pattern of CD45 mRNA expression in said individual, wherein the presence of an abnormal pattern of CD45 mRNA expression characterised by a quantitative increase in the level of expression of the CD45RO transcript is taken as an indication that the subject has an altered immune response capability.

32. A method according to claim 31 wherein the abnormal pattern of CD45 mRNA expression is that associated with the presence of a 138G mutant allele of the gene encoding CD45, wherein detection of the abnormal pattern of CD45 mRNA expression is taken as an indication that the subject has an altered immune response capability, as compared to subjects who do not carry a 138G mutant allele.

33. A method of screening a human subject for susceptibility to viral infection and/or pre-disposition to developing severe disease following viral infection which comprises evaluating the pattern of CD45 protein expression in the subject, wherein the presence of an abnormal pattern of CD45 protein expression characterised as a reduction in the proportion of T lymphocytes expressing the CD45RO isoform but lacking CD45RA expression is taken as an indication that the subject is susceptible to viral infection and/or pre-disposed to developing severe disease following viral infection.

34. A method according to claim 33 wherein the abnormal pattern of CD45 protein expression is that associated with the presence of a 77G mutant allele of the gene encoding CD45, wherein detection of the abnormal pattern of CD45 protein expression is taken as an indication that the subject is more susceptible to viral infection and/or more pre-disposed to developing severe disease following viral infection, as compared to subjects who do not carry a 77G mutant allele.

35. A method according to claim 33 wherein the abnormal pattern of CD45 protein expression is that associated with the presence of a 59A mutant allele of the gene encoding CD45, wherein detection of the abnormal pattern of CD45 protein expression is taken as an indication that the subject is more susceptible to viral infection and/or more pre-disposed to developing severe disease following viral infection, as compared to subjects who do not carry a 59A mutant allele.

36. A method of screening a human subject for susceptibility to viral infection and/or pre-disposition to developing severe disease following viral infection which comprises evaluating the pattern of CD45 protein expression in the subject, wherein the presence of an abnormal pattern of CD45 protein expression characterised by an increase in the proportion of T lymphocytes expressing the CD45RO isoform but lacking CD45RA expression is taken as an indication that the subject is not susceptible to viral infection and/or is pre-disposed to developing less severe disease following viral infection.

37. A method according to claim 36 wherein the abnormal pattern of CD45 protein expression is that associated with the presence of a 138G mutant allele of the gene encoding CD45, wherein detection of the abnormal pattern of CD45 protein expression is taken as an indication that the subject is less susceptible to viral infection and/or pre-disposed to developing less severe disease following viral infection, as compared to subjects who do not carry a 138G mutant allele.

38. A method of screening a human subject for an altered immune response capability, which method comprises evaluating the pattern of CD45 protein expression in said individual, wherein the presence of an abnormal pattern of CD45 protein expression characterized as an increased in the proportion of T lymphocytes expressing the CD45RO isoform but lacking CD45RA expression is taken as an indication that the individual has an altered immune response profile as compared to individuals that do not carry the mutation.

39. The method according to claim 38 wherein the abnormal pattern of CD45 protein expression is that associated with the presence of a 138G mutant allele of the gene encoding CD45, wherein detection of the abnormal pattern of CD45 protein expression is taken as an indication that the subject has an altered immune response capability, as compared to subjects who do not carry a 138G mutant allele.
